(19) United States
(12) Patent Application Publication
(10) Pub. No.: US 2017/0281078 A1
(43) Pub. Date: Oct. 5, 2017

(54) CIRCADIAN RHYTHM EXAMINING DEVICE, CIRCADIAN RHYTHM EXAMINING SYSTEM AND CIRCADIAN RHYTHM EXAMINING METHOD

(71) Applicant: Akiyoshi SHIMURA, Tokyo (JP)

(72) Inventor: Akiyoshi SHIMURA, Tokyo (JP)

(21) Appl. No.: 15/508,303

(22) PCT Filed: Aug. 26, 2015
(86) PCT No.: PCT/JP2015/073966

§ 371 Date: Mar. 2, 2017

(30) Foreign Application Priority Data
Sep. 3, 2014 (JP) 2014-179316

(51) Int. Cl. A61B 5/00; G01B 11/02; A61B 5/107

(52) U.S. Cl. CPC A61B 5/4857; A61B 5/0077; A61B 5/1072; A61B 5/1079; A61B 5/448; A61B 5/443; A61B 5/7278; A61B 5/681; G01B 11/02; A61B 2576/00

(57) ABSTRACT

There are provided an image acquiring unit (122) for acquiring a plurality of images of a skin of an examination subject which is picked up over a plurality of points of time by an image pickup device (104), a body hair specifying unit (123) for specifying body hair for a growth period of the examination subject from the image with regard to each of the images acquired by the image acquiring unit (122), and a circadian rhythm estimating unit 124 for estimating a circadian rhythm of the examination subject based on a temporal change in a length of the body hair for the growth period which is obtained by specifying the body hair for the growth period from each of the images by the body hair specifying unit 123. Consequently, it is possible to estimate the circadian rhythm of the examination subject from these images by simply acquiring the image of the skin of the examination subject without requiring gene measurement, cell culture, frequent blood collection, analysis in special research facilities or the like.

### TECHNICAL FIELD

The present invention relates to a circadian rhythm examining device, a circadian rhythm examining system and a circadian rhythm examining method for examining a circadian rhythm of an organism.

### BACKGROUND ART

In general, a large number of organisms including a human have circadian rhythms (internal clocks) and a gene expresses cyclically based on the circadian rhythm. For this reason, there is observed a time biological phenomenon in which a time taken for cell growth during the day is limited due to each tissue or a mental or physical performance is greatly varied during the day or a time pharmacological phenomenon in which an effect is varied depending on a time zone for use of the same drug, for example.

For instance, a normal myeloid stem cell and a hematopoietic stem cell of a human multiply and are discharged during the day time in the internal clock of the human. In the case in which an anticancer drug is administered to the human in the daytime, therefore, a side effect of myelosuppression tends to be caused in the human. On the other hand, in the case in which the anticancer is administered to the human at night, the side effect in the human can be reduced. In another example, moreover, the digestive tract function of a human is deteriorated at midnight in the interior clock of the human. For this reason, the human tends to have dull feeling in the stomach when the human eats or drinks at midnight.

From the foregoing, it is preferable to specify a proper time zone for carrying out an activity by a human or administering a drug to the human based on a circadian rhythm of the human in order to obtain excellent effects through various activities of the human (for example, labors, exercises, meals, sleeps and the like) and drug administration. By performing various activities or drug administration in the specified proper time zone, it is possible to enhance an effect for improving a lifestyle habit of a human or to increase a therapeutic effect for a human.

However, the circadian rhythm has an individual difference. For example, some humans have an internal clock of morning in the general morning (that is, some humans who rarely have a phase shift of the circadian rhythm) and others have an internal clock of night (that is, humans having a great phase shift of the circadian rhythm). In order to specify the proper time zone for causing a human to carry out an activity or administering a drug to a human, accordingly, it is necessary to grasp a peculiar a circadian biorhythm to the human.

For example, the following Patent Document 1 discloses a method of estimating a circadian rhythm of an examination subject based on a temporal change in an expression level of a clock gene in a hair follicle stem cell adhering to body hair (hair, beard or arm hair) of the examination subject, thereby detecting a phase shift of the circadian rhythm.

Patent Document 1: Japanese Laid-Open Patent Publication No. 2009-27952

### DISCLOSURE OF THE INVENTION

However, it is conventionally necessary to perform gene measurement, cell culture, frequent blood collection, analysis in special research institutions or the like in order to grasp a circadian rhythm of an organism. For this reason, there is conventionally a problem in that it is impossible to easily grasp the circadian rhythm of the organism. For example, referring to the technique disclosed in the Patent Document 1, it is necessary to acquire a hair follicle cell plural times and to extract RNA by using a commercially available RNA extraction kit every acquirement of a hair follicle cell, and to then perform reverse transcription, and furthermore, to measure a gene expression level by using a real-time PCR device. For this reason, referring to the technique disclosed in the Patent Document 1, it is impossible to easily grasp a circadian rhythm.

The present invention has been made to solve the problem and has an object to enable a circadian rhythm of an organism to be grasped easily.

In order to attain the object, the present invention serves to acquire a plurality of images of a skin of an organism which is picked up over a plurality of points of time by an image pickup device and to specify body hair for a growth period of the organism from the image with regard to each of the acquired images. The present invention serves to estimate a circadian rhythm of an organism based on a temporal change in a length of body hair for a growth period which is obtained by specifying the body hair for the growth period from each of images.

According to the present invention having the structure described above, it is possible to estimate a circadian rhythm of an organism from a plurality of images of a skin of the organism by simply acquiring the images plural times without requiring gene measurement, cell culture, frequent blood collection, analysis in special research institutions or the like. According to the present invention, therefore, it is possible to easily grasp the circadian rhythm of the organism.

### BRIEF DESCRIPTION OF THE DRAWINGS

FIG. 1 is a view showing an example of a structure of a circadian rhythm examining device according to a first embodiment of the present invention.

FIG. 2 is a chart showing an example of a circadian rhythm to be estimated by the circadian rhythm examining device according to a first embodiment of the present invention.

FIG. 3 is a view showing an example of a device structure of the circadian rhythm examining device according to the first embodiment of the present invention.

FIG. 4 is a view showing an example of arrangement of an irradiating device and an image pickup device according to the first embodiment of the present invention.

FIG. 5 is a chart showing light absorption characteristics of oxyhemoglobin and melanin.

FIG. 6 is a diagram for explaining a state of in which light irradiated on a skin is reflected.

FIG. 7 is a block diagram showing an example of a functional structure of a control module according to the first embodiment of the present invention.

FIG. 8 is a view showing an example of a skin image picked up by the image pickup device according to the first embodiment of the present invention.

FIG. 9 is a flowchart showing an example of a processing to be performed by the circadian rhythm examining device according to the first embodiment of the present invention.

FIG. 10 is a view showing an example of a display screen to be displayed by the circadian rhythm examining device according to the first embodiment of the present invention.

FIG. 11 is a view showing an example of a structure of a circadian rhythm examining system according to a second embodiment of the present invention.

FIG. 12 is a block diagram showing an example of a functional structure of the circadian rhythm examining system according to the second embodiment of the present invention.

FIG. 13 is a view showing an example of a structure of a circadian rhythm examining system according to a third embodiment of the present invention.

FIG. 14 is a block diagram showing an example of a functional structure of the circadian rhythm examining system according to the third embodiment of the present invention.

FIG. 15 is a chart showing an example of synthesis of a waveform of a circadian rhythm through a server according to the third embodiment of the present invention.

FIG. 16 is a chart showing an example of a method of estimating a circadian rhythm using a recognition pattern according to the third embodiment of the present invention.

### BEST MODE FOR CARRYING OUT THE INVENTION

### First Embodiment

[Example of Structure of Circadian Rhythm Examining Device 100]

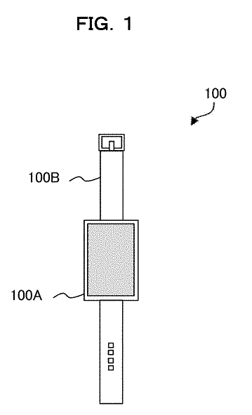

A first embodiment according to the present invention will be described below with reference to the drawings. FIG. 1 is a view showing an example of a structure of a circadian rhythm examining device 100 according to the first embodiment of the present invention. The circadian rhythm examining device 100 is attached to a skin surface of an examination subject (an example of an organism within the claims) to examine a circadian rhythm of the examination subject.

As shown in FIG. 1, the circadian rhythm examining device 100 according to the first embodiment includes a a body 100A and a belt 100B. Consequently, the circadian rhythm examining device 100 takes a shape of a watch or the like. In the circadian rhythm examining device 100, the belt 100B is wound around a hand or leg of an examination subject so that the body 100A can be fixed to a skin surface of the hand or leg of the examination subject.

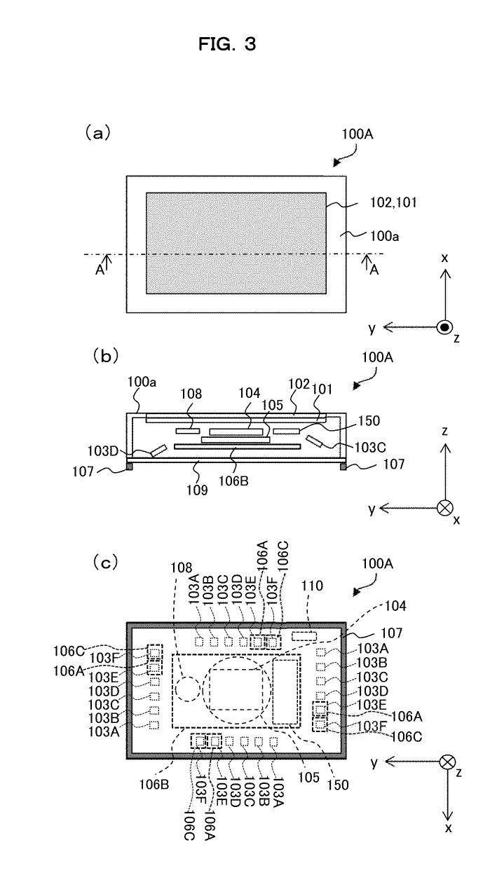

The circadian rhythm examining device 100 includes an irradiating device 103 and an image pickup device 104 (see FIG. 3). Consequently, the circadian rhythm examining device 100 can irradiate light on the skin surface of the examination subject by the irradiating device 103 and can pick up an image of a skin (hereinafter referred to as a “skin image”) of the examination subject by the image pickup device 104 in a state in which it is attached to the skin surface of the examination subject.

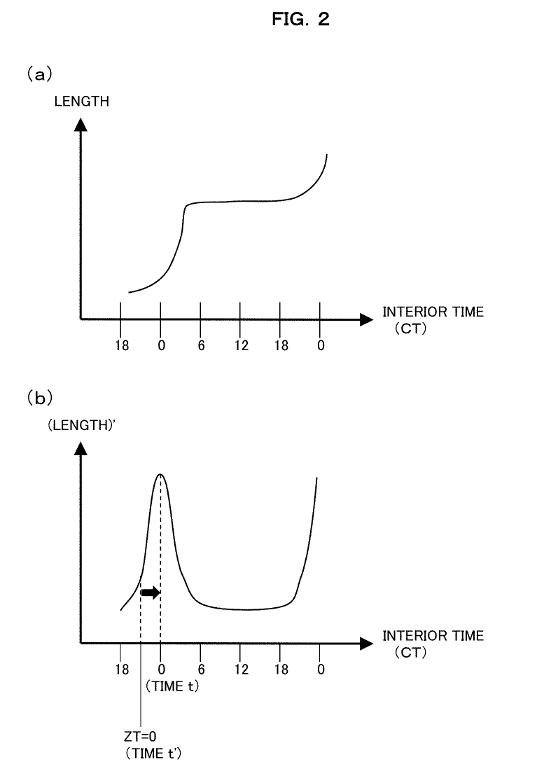

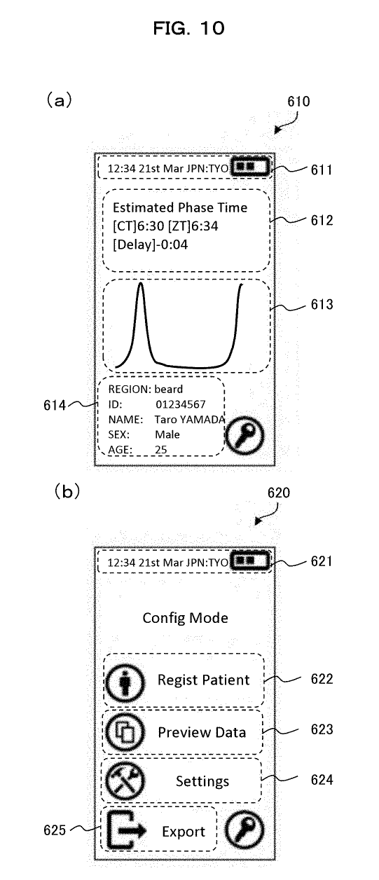

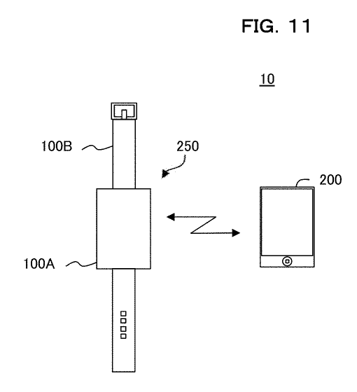

Moreover, the circadian rhythm examining device 100 includes a control module 150 (see FIG. 3). The control module 150 specifies body hair for a growth period from each of skin images picked up over a plurality of points of time by the image pickup device 104, thereby obtaining a temporal change in a length of the body hair shown in FIG. 2(a). Then, the control module 150 estimates a circadian rhythm of the examination subject shown in FIG. 2(b) based on the temporal change. Furthermore, the control module 150 calculates a phase shift of the circadian rhythm of the examination subject based on an estimated circadian rhythm and a time of a light-dark condition which is predetermined. Then, the control module 150 causes a display 101 (see FIG. 3) to display the estimated circadian rhythm and the phase shift of the circadian rhythm which is calculated through a display screen shown in FIG. 10(a).

[Example of Circadian Rhythm]

FIG. 2 is a chart showing an example of a circadian rhythm to be estimated by the circadian rhythm examining device 100 according to the first embodiment of the present invention.

In the circadian rhythm examining device 100, a plurality of measurement times for respective predetermined times (for example, every 30 minutes, every hour or the like) is predetermined, for example. The circadian rhythm examining device 100 picks up a skin image by the image pickup device 104 every time the measurement time arrives. In other words, the image pickup device 104 picks up a plurality of skin images over a plurality of measurement times. The control module 150 detects a length of body hear for a growth period of the examination subject from each of the skin images.

Then, the control module 150 specifies a temporal change in the lengths of the body hair for the growth period of the examination subject shown in FIG. 2(a) based on the lengths of the body hair for the growth period of the examination subject which are detected plural times. Furthermore, the control module 150 differentiates the temporal change in the length of the body hair for the growth period of the examination subject which is specified, thereby specifying a temporal change in a growth speed of the body hair of the examination subject which is shown in FIG. 2(b) to estimate the temporal change in the growth speed of the body hair as the circadian rhythm of the examination subject. The reason is that it can be supposed that the growth speed of the body hair for the growth period of the examination subject is changed based on the circadian rhythm of the examination subject.

In the circadian rhythm examining device 100, moreover, the time of the light-dark condition in chronobiology is predetermined. The control module 150 calculates a phase shift of a circadian rhythm for the time of the light-dark condition based on the estimated circadian rhythm and the time of the light-dark condition which is predetermined. More specifically, the control module 150 sets an interior time CT (Circadian Time) of the examination subject=0 at a time t that a peak (that is, a peak of the growth speed of the body hair) is reached in the estimated circadian rhythm as shown in FIG. 2(b). At this time, it is assumed that a time ZT (Zeitgeber Time)=0 in the light-dark condition is preset at a time t′ as the time of the light-dark condition. In this case, the control module 150 calculates a difference between the time t setting the interior time CT=0 and the time t′ setting the time ZT=0 in the light-dark condition as the phase shift of the circadian rhythm.

Herein, the time ZT=0 in the light-dark condition implies a start time for a light condition (sunrise in a natural condition). For example, In the case of Vernal Equinox Day and Autumnal Equinox Day, ZT=0 is set at around six o'clock in the morning. In other words, the time t′ that the time ZT=0 is set is varied with a date. Therefore, it is preferable to store the time t′ that the time ZT=0 is set every date and to use the time t′ corresponding to a current date when obtaining the phase shift of the circadian rhythm.

On the other hand, the interior time CT=0 implies an activity starting time in the internal clock depending on the circadian rhythm. After the activity starting time, an organism performs activities in accordance with the circadian rhythm. For example, the organism wakes up with around CT=0, a beard or the like grows at the same time and the activity of a digestive tract is started. Moreover, a deep body temperature reaches a peak at around CT=8 to 12 and the activity of the digestive tract is reduced at around CT=16. After falling sleep, furthermore, the deep body temperature is made the lowest at around CT=18 to 0 so that the slow wave sleep (sound sleep) is generated.

In the case in which the time t′ that ZT=0 is set and the time t that CT=0 is set are coincident with each other, accordingly, there is possessed a circadian rhythm for starting an activity simultaneously with sunrise. In this case, therefore, the control module 150 decides that the phase shift of the circadian rhythm is not made. However, the circadian rhythm has a great individual difference and the time t′ that ZT=0 is set is not coincident with the time t that CT=0 is set in most cases. In these cases, the control module 150 decides that the phase shift of the circadian rhythm is made.

For example, in the example shown in FIG. 2(b), it is indicated that the time t that the internal clock CT=0 is set is delayed by three hours from the time t′ that the time ZT=0 in the light-dark condition is set. In other words, the estimated circadian rhythm implies that a shift (delay) of a phase corresponding to three hours is made from the time t of the light-dark condition. It is very important to thus measure the circadian rhythm of the organism and the phase shift thereof in chronopharmacology, chronotherapeutics, a treatment for sleep disorders or the like.

Thus, the circadian rhythm examining device 100 according to the first embodiment can estimate the circadian rhythm of the examination subject and the phase shift thereof from a plurality of skin images obtained by simply picking up the skin image plural times. Therefore, the circadian rhythm examining device 100 according to the first embodiment can easily grasp the circadian rhythm of the examination subject and the phase shift thereof.

[Device Structure of Circadian Rhythm Examining Device 100]

FIG. 3 is a view showing an example of a device structure of the circadian rhythm examining device 100 according to the first embodiment of the present invention. FIG. 3(a) is a front view showing the body 100A of the circadian rhythm examining device 100. Moreover, FIG. 3(b) is a sectional side view showing the body 100A of the circadian rhythm examining device 100 (an A-A sectional view of the body 100A illustrated in FIG. 3(a)). Furthermore, FIG. 3(c) is a rear view showing the body 100A of the circadian rhythm examining device 100.

As shown in FIG. 3, the circadian rhythm examining device 100 has the body 100A including the display 101, a touch panel 102, the irradiating device 103, the image pickup device 104, a macro lens 105, a first polarizing filter 106A, a second polarizing filter 106B, a third polarizing filter 106C, a shielding member 107, a battery 108, a cover 109, a current position detecting device 110 and the control module 150. These components (excluding the shielding member 107 and the cover 109) are accommodated in a housing 100a of the body 100A. The cover 109 is attached to the back side of the housing 100a so that aback face (an external surface opposed to a skin surface) of the housing 100a is formed. Moreover, the shielding member 107 is attached to a surface of the cover 109.

(Housing 100a)

The housing 100a is a container-shaped member. Although the housing 100a takes a shape of a rectangular parallelepiped having a back face opened in an example shown in FIG. 3, the shape of the housing 100a is not restricted thereto. For example, the housing 100a may take a curved shape along the skin surface of the examination subject. Alternatively, the housing 100a formed by a flexible material may be used to take a shape of a plate in a normal state and to be deformable so as to be curved along the skin surface of the examination subject in attachment.

The housing 100a is formed by a material having a light shielding property except for the back face thereof (a surface on a side in a negative direction of a z axis in the drawing) in such a manner that light emitted from an outside does not enter an inner part. The housing 100a is attached onto the skin surface of the examination subject in such a manner that the back face is opposed to the skin surface of the examination subject. In other words, the circadian rhythm examining device 100 irradiates light on the skin of the examination subject from the back face side of the housing 100a, and furthermore, picks up a skin image at the back face side of the housing 100a. For this reason, the cover 109 formed by a material having optical transparency (for example, plastic, silicon, acryl or the like) is used for the back face of the housing 100a.

(Display 101)

The display 101 is provided on the surface side of the housing 100a (a side in a positive direction of the z axis in the drawing) and displays various information (mainly, an examination result of a circadian rhythm). For example, a liquid crystal display, an organic EL display or the like is used for the display 101.

(Touch Panel 102)

The touch panel 102 is an example of an input device for inputting various information. The touch panel 102 is provided on the surface side of the housing 100a and is arranged in superposition on the surface of the display 101. The circadian rhythm examining device 100 may be provided with another input device (for example, a button or the like) in addition to the touch panel 102 or in place of the touch panel 102.

(Irradiating Device 103)

The irradiating device 103 irradiates light on the skin of the examination subject. The irradiating device 103 has an irradiation surface at the back face side of the housing 100a. Consequently, the irradiating device 103 can irradiate light on the skin of the examination subject when the body 100A is attached to the skin surface of the examination subject. The irradiating device 103 includes a luminous body such as an LED.

As shown in FIG. 3(c), plural sets (four sets in the drawing to which the present invention is not restricted) of irradiating devices 103A to 103F are provided in the housing 100a. The respective irradiating devices 103A to 103F are arranged every set in four directions around the image pickup device 104 in such a manner that an illuminance of light on the skin surface is made as uniform as possible. Consequently, a shadow can be prevented from being made in photographing according to the principle of a shadowless lamp. Also in the case in which a plurality of body hair overlap each other as seen from a certain optional viewpoint, moreover, light can be irradiated on the respective body hair. Each of the irradiating devices 103A to 103F can irradiate light independently of the other irradiating devices by control of the control module 150.

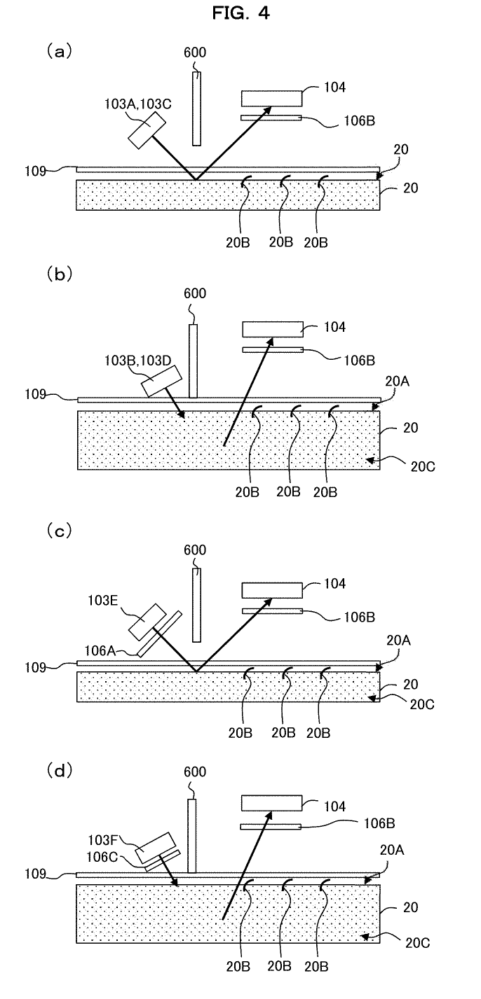

The irradiating device 103A is the irradiating device 103 which emits white light (or broad spectral light) and is arranged in a separated position from the skin surface in the housing 100a (see FIG. 4(a)). The irradiating device 103B is the irradiating device 103 which emits white light (or broad spectral light) and is arranged in a close position to the skin surface in the housing 100a (see FIG. 4(b)).

The irradiating device 103C is the irradiating device 103 which emits light having a specific wavelength and is arranged in a separated position from the skin surface in the housing 100a (see FIG. 4(a)). The irradiating device 103D is the irradiating device 103 which emits light having a specific wavelength and is arranged in a close position to the skin surface in the housing 100a (see FIG. 4(b)).

The irradiating device 103E is the irradiating device 103 which emits white light (or broad spectral light) and is arranged in a separated position from the skin surface in the housing 100a (see FIG. 4(c)). Moreover, the irradiating device 103E is covered with the first polarizing filter 106A.

The irradiating device 103F is the irradiating device 103 which emits light having an optional wavelength and is arranged in a close position to the skin surface in the housing 100a (see FIG. 4(d)). Moreover, the irradiating device 103F is covered with a third polarizing filter 106C.

The white light is obtained by equally mixing light having respective colors in visible rays (blue light (400 to 500 nm), green light (500 to 600 nm), red light (600 to 700 nm)) and does not give color feeling. The broad spectral light is light which is represented by an incandescent lamp, has no specific light wavelength and has abroad spectrum. For example, a light source to be used in emission of the white light or the broad spectral light includes a small-sized incandescent lamp, a white LED, an organic white EL, a halogen lamp, a xenon lamp, a white laser and the like.

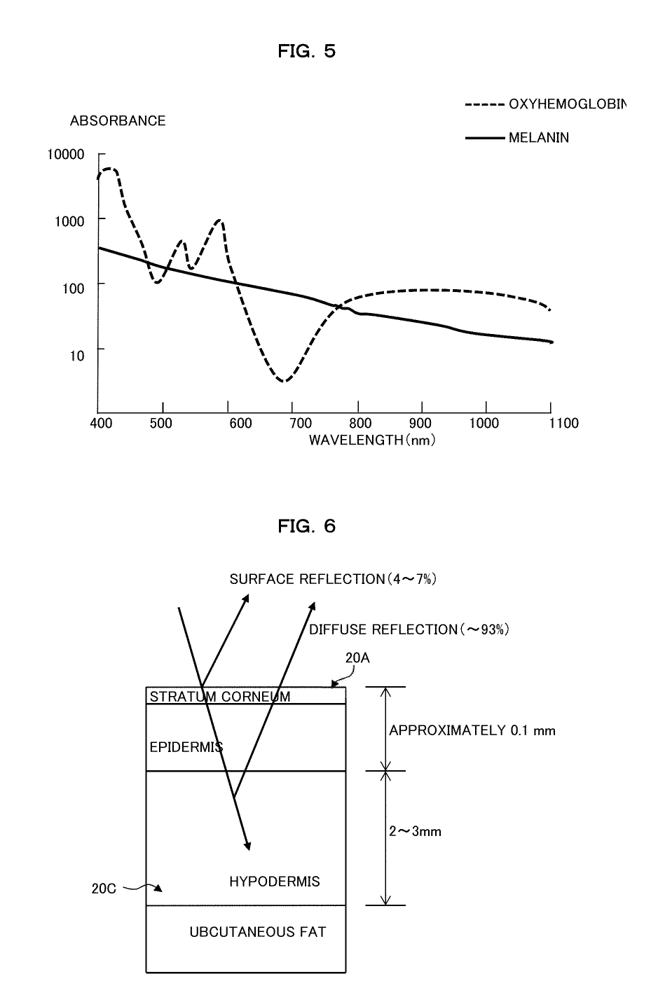

On the other hand, in the first embodiment, the light having a specific wavelength has such a characteristic that it can easily be absorbed into melanin (a main element constituting a color of body hair) and is absorbed into hemoglobin (a main element constituting a color of a skin) with difficulty. For instance, Light having this characteristic includes light in the vicinity of 500 nm (for example, an argon laser beam), light in the vicinity of 690 nm (for example, a ruby laser beam) and the like as shown in FIG. 5. The circadian rhythm examining device 100 according to the first embodiment can obtain a skin image having body hair emphasized by using the light having the specific wavelength. Consequently, the circadian rhythm examining device 100 can easily specify the body hair from the skin image.

It is preferable to use, for the irradiating devices 103A, 103B and 103E, a light source capable of emitting white light or broad spectral light which covers a detection region of an image pickup element of the image pickup device 104. For example, in the case in which a CCD (Charge Coupled Device) having a detection region of 300 to 800 nm is used for the image pickup element of the image pickup device 104, it is preferable to use a light source capable of emitting white light or broad spectral light which covers a wavelength region of 300 to 800 nm.

(Image Pickup Device 104)

The image pickup device 104 picks up a skin image. The image pickup device 104 has an image pickup surface on the back face side of the housing 100a (the side in the negative direction of the z axis in the drawing). Consequently, the image pickup device 104 can pick up the skin image through light reflected from a skin when the body 100A is attached to the skin surface of the examination subject. The image pickup device 104 includes an image pickup element such as a CCD or a CMOS (Complementary Metal Oxide Semiconductor) or the like, for example.

In the first embodiment, the body hair is specified from the skin image picked up by the image pickup device 104. For this reason, it is preferable to use the image pickup device 104 having a sufficient image resolution. For example, the body hair has a thickness of approximately 30 to 60 μm for hair of arms and legs which is greater than 20 μm. Therefore, if the area of the skin for image pickup is set to be 20 mm×20 mm, for example, there is required the image pickup device 104 capable of detecting an object in a size of one thousandth of a length of one side. In this case, it is possible to sufficiently detect a microconstituent such as body hair by using the image pickup device 104 having an image resolution of 1000000 pixels or more. In the case in which the area of the skin for image pickup is different from the example (20 mm×20 mm), it is preferable to change the image resolution of the image pickup device 104 from the example (1000000 pixels) if necessary.

(Macro Lens 105)

The macro lens 105 is arranged on an outside of the image pickup surface of the image pickup device 104 (the skin surface side). The macro lens 105 shortens a focal length of the image pickup device 104 to form an image through light reflected from a skin over the image pickup surface of the image pickup device 104. If the image pickup device 104 has an image pickup element which is sufficiently larger than the image pickup range, it is also possible to employ a structure in which the macro lens 105 is not provided.

(First Polarizing Filter 106A)

The first polarizing filter 106A is disposed in front of the irradiation surface of the irradiating device 103E to cover the irradiating device 103E. The first polarizing filter 106A restricts light to be irradiated on the skin of the examination subject in the light emitted from the irradiating device 103E to light having a predetermined vibrating direction.

(Second Polarizing Filter 106B)

The second polarizing filter 106B is disposed in front of the image pickup surface of the image pickup device 104 to cover the image pickup device 104. The second polarizing filter 106B restricts light to be incident on the image pickup device 104 in the light reflected from the skin of the examination subject to light having a predetermined vibrating direction.

(Third Polarizing Filter 106C)

The third polarizing filter 106C is disposed in front of the irradiation surface of the irradiating device 103F to cover the irradiating device 103F. The third polarizing filter 106C restricts light to be irradiated on the skin of the examination subject in the light emitted from the irradiating device 103F to light having a predetermined vibrating direction.

The first polarizing filter 106A and the second polarizing filter 106B are arranged with polarizing directions in parallel with each other. Consequently, a vibrating direction of light to be transmitted through the first polarizing filter 106A and irradiated on the skin of the examination subject and a vibrating direction of light to be transmitted through the second polarizing filter 106B and incident on the image pickup device 104 are made parallel with each other.

Moreover, the third polarizing filter 106C and the second polarizing filter 106B arranged in such a manner that polarizing directions are orthogonal to each other. Consequently, a vibrating direction of light to be transmitted through the third polarizing filter 106C and irradiated on the skin of the examination subject and the vibrating direction of the light to be transmitted through the second polarizing filter 106B and incident on the image pickup device 104 are made orthogonal to each other.

[Specific Example of Method of Picking Up Skin Image]

With reference to FIGS. 4 to 6, description will be given to a specific example of the method of picking up a skin image by the circadian rhythm examining device 100 according to the first embodiment. FIG. 4 is a view showing an example of arrangement of the irradiating device 103 and the image pickup device 104 according to the first embodiment of the present invention. FIG. 5 is a chart showing light absorption characteristics of oxyhemoglobin and melanin. FIG. 6 is a diagram for explaining a state in which light irradiated on a skin is reflected.

Referring to the circadian rhythm examining device 100 according to the first embodiment, it is possible to selectively apply, as a method of picking up a skin image of a skin 20 of the examination subject, one or more of first to sixth image pickup methods which will be explained below by setting to a set information storing unit 111 which will be described later.

(First and Second Image Pickup Methods)

The first image pickup method serves to pick up the skin image of the skin 20 through the image pickup device 104 while irradiating white light or broad spectral light on the skin 20 by the irradiating device 103A which is separated from a skin surface 20A. The second image pickup method serves to pick up the skin image of the skin 20 through the image pickup device 104 while irradiating white light or broad spectral light on the skin 20 by the irradiating device 103B which is close to the skin surface 20A.

According to the first and second image pickup methods, the skin 20 and body hair 20B have absorbances which are almost equal to each other for the white light or the broad spectral light irradiate from the irradiating devices 103A and 103B. Therefore, it is possible to obtain a skin image in which the image of the skin 20 containing more hemoglobin pigments and the image of the body hair 20B containing more melanin pigments have almost equal brightnesses.

In particular, the first image pickup method uses the irradiating device 103A disposed in a separated position from the skin surface 20A (a first irradiating device described in claims) as shown in FIG. 4(a). Therefore, the image pickup device 104 can obtain more light reflected from the skin surface 20A. Accordingly, there is obtained a skin image with projection of the same image as an image recognized by a man or a woman when seeing the skin surface 20A under normal light. In this case, it is possible to better observe the body hair 20B exposed to the skin surface 20A from the skin image picked up by the image pickup device 104.

On the other hand, the second image pickup method uses the irradiating device 103B arranged in a close position to the skin surface 20A (a second irradiating device described in claims) as shown in FIG. 4(b). Therefore, the image pick-up device 104 can obtain more light reflected from a deep skin layer 20C. In this case, it is possible to observe the body hair 20B present in and beneath an epidermis in addition to the body hair 20B exposed to the skin surface 20A from the skin image picked up by the image pickup device 104.

(Third Image Pickup Method)

The third image pickup method first picks up a skin image (a first image) of the skin 20 by the image pickup device 104 while irradiating white light or broad spectral light on the skin 20 by the irradiating device 103A which is separated from the skin surface 20A. Next, the method picks up a skin image (a second image) of the skin 20 by the image pickup device 104 while irradiating, on the skin 20, light having a specific wavelength which is easily absorbed into melanin and is hard to be absorbed into hemoglobin (for example, light in the vicinity of 500 nm or light in the vicinity of 690 nm) by the irradiating device 103C separated from the skin surface 20A.

(Fourth Image Pickup Method)

A fourth image pickup method first picks up a skin image (a first image) of the skin 20 by the image pickup device 104 while irradiating white light or broad spectral light on the skin 20 by the irradiating device 103B which is close to the skin surface 20A. Next, the method picks up a skin image (a second image) of the skin 20 by the image pickup device 104 while irradiating, on the skin 20, light having a specific wavelength which is easily absorbed into melanin and is hard to be absorbed into hemoglobin (for example, light in the vicinity of 500 nm or light in the vicinity of 690 nm) by the irradiating device 103D which is close to the skin surface 20A.

According to the third and fourth image pickup methods, the absorbance of the white light or the broad spectral light is almost equal for the skin 20 and the body hair 20B when the light is irradiated on the skin surface 20A. For this reason, it is possible to obtain, as the first image, a skin image in which the image of the skin 20 containing a large number of hemoglobin pigments and the image of the body hair 20B containing a large number of melanin pigments have brightnesses that are almost equal to each other.

According to the third and fourth image pickup methods, moreover, the absorbance of light having a specific wavelength in irradiation of the light having a specific wavelength on the skin 20 is greatly different between the skin 20 and the body hair 20B. Therefore, it is possible to obtain, as the second image, an image in which a brightness is greatly different between the image of the skin 20 containing a large number of hemoglobin pigments and the image of the body hair 20B containing a large number of melanin pigments.

In particular, the third image pickup method uses the irradiating device 103C arranged in a separated position from the skin surface 20A (the first irradiating device described in claims) as shown in FIG. 4(a). Therefore, the image pick-up device 104 can obtain more light reflected from the skin surface 20A. In this case, it is possible to better observe the body hair 20B exposed to the skin surface 20A from the skin image picked up by the image pickup device 104.

On the other hand, the fourth image pickup method uses the irradiating device 103D arranged in a close position to the skin surface 20A (the second irradiating device described in claims) as shown in FIG. 4(b). Therefore, the image pick-up device 104 can obtain more light reflected from the deep skin layer 20C. In this case, it is possible to observe the body hair 20B present in and beneath the epidermis in addition to the body hair 20B exposed to the skin surface 20A from the skin image picked up by the image pickup device 104.

As shown in FIGS. 4(a) and 4(b), the second polarizing filter 106B is arranged in front of the image pickup device 104. Referring to the first to fourth image pickup methods, however, it is not necessary to limit a vibrating direction of light to be incident on the image pickup device 104. For this reason, in the case of the first to fourth image pickup methods, it is preferable to configure the second polarizing filter 106B so as to enable turn-OFF a function for limiting the vibrating direction of light by utilization of ON/OFF of a polarizing function through a liquid crystal or the like, for example. Alternatively, the image pickup device 104 having no polarizing filter arranged in front may be further provided for the first to fourth image pickup methods.

(Fifth Image Pickup Method)

A fifth image pickup method serves to irradiate light (white light or broad spectral light) having a specific vibrating direction with respect to the skin surface 20A, thereby picking up the skin image of the skin 20 through reflecting light having a parallel vibrating direction with the light by the image pickup device 104. More specifically, as shown in FIG. 4(c), the irradiating device 103E is caused to irradiate light. The light emitted from the irradiating device 103E is transmitted through the first polarizing filter 106A. Consequently, only a component having the specific vibrating direction is irradiated on the skin 20. Light reflected from the skin surface 20A is transmitted through the second polarizing filter 106B. Consequently, only a component having a parallel vibrating direction with the light irradiated on the skin 20 is incident on the image pickup device 104. Thus, the image pickup device 104 can pick up the skin image of the skin through reflected light having the parallel vibrating direction with the light irradiated on the skin 20.

According to the fifth image pickup method, a part (approximately 4 to 7%) of the light irradiated on the skin 20 is reflected from the skin surface 20A and another part is diffused and reflected from the deep skin layer 20C as shown in detail in FIG. 6. In other words, the light reflected from the skin 20 contains the light reflected from the skin surface 20A and the light reflected from the deep skin layer 20C. Because the light reflected from the skin surface 20A has a vibrating direction unchanged (that is, the reflected light is parallel with the light irradiated on the skin 20), it is transmitted through the second polarizing filter 106B and is then incident on the image pickup device 104. On the other hand, the light reflected from the deep skin layer 20C repeats irregular reflection • interference • absorption • excitation together with an internal structure or a substance in an inner part so that polarized light disappears. Therefore, the reflected light is removed by the second polarizing filter 106B. For this reason, the image pickup device 104 can pick up a skin image obtained by the light reflected from the skin surface 20A (that is, a skin image having a large amount of information related to an uneven state of the skin surface 20A).

In the fifth image pickup method, moreover, it is also possible to provide two or more image pickup devices 104 having different inclinations of the image pickup direction with respect to the skin surface 20A. In this case, it is possible to pick up the same body hair in a plurality of viewpoint directions by the principle of stereoscopic vision. Therefore, it is possible to detect a cubic shape of the body hair (that is, a contour of the body hair) in the viewpoint directions.

(Sixth Image Pickup Method)

A sixth image pickup method serves to irradiate light with an optional wavelength having a specific vibrating direction with respect to the skin 20, thereby picking up the skin image of the skin 20 through reflected light having an orthogonal vibrating direction to the light by the image pickup device 104. More specifically, as shown in FIG. 4(d), light is irradiated from the irradiating device 103F. The light emitted from the irradiating device 103F is transmitted through the third polarizing filter 106C. Consequently, only a component having the specific vibrating direction is irradiated on the skin 20. In light reflected from the skin 20, only a component having an orthogonal vibrating direction to the light irradiated on the skin 20 is incident on the image pickup device 104 by the second polarizing filter 106B. Thus, the image pickup device 104 can pick up the skin image through reflected light having the orthogonal vibrating direction to the light irradiated on the skin 20.

According to the sixth image pickup method, the light reflected from the skin 20 contains the light reflected from the skin surface 20A and the light reflected from the deep skin layer 20C. Because the light reflected from the skin surface 20A has a vibrating direction unchanged (that is, the light is parallel with the light irradiated on the skin 20), it is removed by the second polarizing filter 106B. On the other hand, since the light reflected from the deep skin layer 20C is scattered light and has a large number of non-polarizing components (that is, the reflected light is not parallel with the light irradiated on the skin 20), it is transmitted through the second polarizing filter 106B and is incident on the image pickup device 104. Therefore, the image pickup device 104 can pick up a skin image through the light reflected from the deep skin layer 20C (that is, a skin image having a large amount of information related to a state of an inner part of the skin).

Referring to the first to sixth image pickup methods, moreover, a shielding plate 600 having a light shielding property is provided between the irradiating device 103 and the image pickup device 104 as shown in FIGS. 4(a) to 4(d). Thus, it is possible to prevent the light irradiated from the irradiating device 103 from being directly incident on the image pickup device 104. Consequently, unnecessary light for the light reflected from the skin 20 can be prevented from being mixed. Therefore, the skin image to be picked up by the image pickup device 104 can be made clearer.

In the first to sixth image pickup methods, moreover, it is preferable to moderately incline an emitting direction of the irradiating device 103 toward the optical axis side of the image pickup device 104 as shown in FIGS. 4(a) to 4(d). Consequently, the image pickup device 104 can easily detect the light reflected from the skin surface 20A or the deep skin layer 20C.

The circadian rhythm examining device 100 serves to use any of the first to sixth image pickup methods which is set to the set information storing unit 111 in the examination of the circadian rhythm of the examination subject. In particular, the circadian rhythm examining device 100 can enhance detection precision in the body hair of the examination subject by executing a plurality of image pickup methods in combination based on the setting of the set information storing unit 111.

In particular, it is preferable to pick up both a skin image through the light reflected from the skin surface 20A and a skin image through the light reflected from the deep skin layer 20C by executing a plurality of image pickup methods in combination. The skin image through the light reflected from the skin surface 20A can be picked up by the third and fifth image pickup methods. A skin image through the light reflected from the deep skin layer 20C can be picked up by the second, fourth and sixth image pickup methods.

Since the second, fourth and sixth image pickup methods can specify the body hair present in the deep skin layer 20C, they are preferably executed in combination with any of the first, third and fifth image pickup methods capable of specifying the body hair 20B present o the skin surface 20A. Consequently, it is possible to specify both the body hair 20B present on the skin surface 20A and the body hair 20B present on the deep skin layer 20C.

In addition, the image pickup methods are combined variously. For example, skin images may be picked up by all of the first to sixth image pickup methods to specify the body hair 20B from each of the skin images. In this case, the body hair 20B specified in all of the skin images may be determined as the body hair 20B to be an estimation target of a circadian rhythm.

In the case in which a skin image is picked up by one of the image pickup methods and the body hair 20B cannot be detected from the skin image, a skin image may be picked up by the other image pickup method to try the detection of the body hair 20B from the skin image.

It is also possible to configure the circadian rhythm examining device 100 so as to enable execution of only a part of the first to sixth image pickup methods particularly in the case in which a cost is to be reduced, the case in which an effective image pickup method for an examination subject is restricted, or the like. In other words, a device for the image pickup method which is not executed (for example, an irradiating device, a polarizing filter or the like) may be omitted from the circadian rhythm examining device 100.

(Shielding Member 107)

The shielding member 107 is provided on the surface and along an outer peripheral edge portion of the cover 109 configuring the back face of the housing 100a. The shielding member 107 shields light from an outside in such a manner that the light from the outside does not enter an inner part of the housing 100a in a clearance between the back face of the housing 100a and the skin surface by adherence to the skin surface of the examination subject when the body 100A is attached to the skin surface. For this reason, the shielding member 107 is formed by a material having a light shielding property. Moreover, the shielding member 107 can also prevent the housing 100a from being shifted from the skin surface by friction force with the skin surface. It is preferable that the shielding member 107 should be formed by a flexible material (for example, a rubber material) so as to enable enhancement in adhesion to the skin surface by deformation along the skin surface.

(Battery 108)

The battery 108 supplies power for operating the circadian rhythm examining device 100. For example, a lithium ion battery, a nickel-cadmium battery, an alkaline battery, a manganese dioxide battery or the like is used for the battery 108.

(Cover 109)

The cover 109 is a transparent plate-shaped member which covers the back face side of the housing 100a. The cover 109 is attached to the back face side of the housing 100a to configure the back face of the housing 100a. Light emitted from the irradiating device 103 in the housing 100a is transmitted through the cover 109, and is thus emitted to the outside of the housing 100a and is irradiated on the skin of the examination subject. Moreover, light reflected from the skin of the examination subject is transmitted through the cover 109 and is thus incident on the inner part of the housing 100a and is incident on the image pickup device 104. Furthermore, the cover 109 plays apart in prevention of mixture of foreign substances into the inner part of the housing 100a or contact of internal components of the housing 100a with the skin surface of the examination subject.

Herein, it is necessary to cause the back face side of the body 100A to be close to the skin surface in order to pick up an excellent skin image by the image pickup device 104. At this time, there is a fear that a sebum might adhere to the surface of the cover 109 from the skin surface. In the case in which the sebum adheres to the surface of the cover 109, there is a possibility that the light transmitted through the cover 109 might be badly influenced (for example, be subjected to irregular reflection or the like), resulting in deterioration in image quality of the skin image to be picked up by the image pickup device 104. In the circadian rhythm examining device 100 according to the first embodiment, therefore, the surface of the cover 109 is subjected to a processing for preventing the adhesion of the sebum (for example, fluorine coating or the like).

In the case in which the housing 100a takes a curved shape along the skin surface, it is preferable to correspondingly cause the cover 109 to take a curved shape along the skin surface. In the case in which a material which can be deformed along the skin surface is used for the housing 100a, moreover, it is preferable to correspondingly use a material capable of deforming the cover 109 along the skin surface.

(Current Position Detecting Device 110)

The current position detecting device 110 serves to detect a current position of the circadian rhythm examining device 100. The current position detecting device 110 includes a GPS (Global Positioning System), for example.

(Control Module 150)

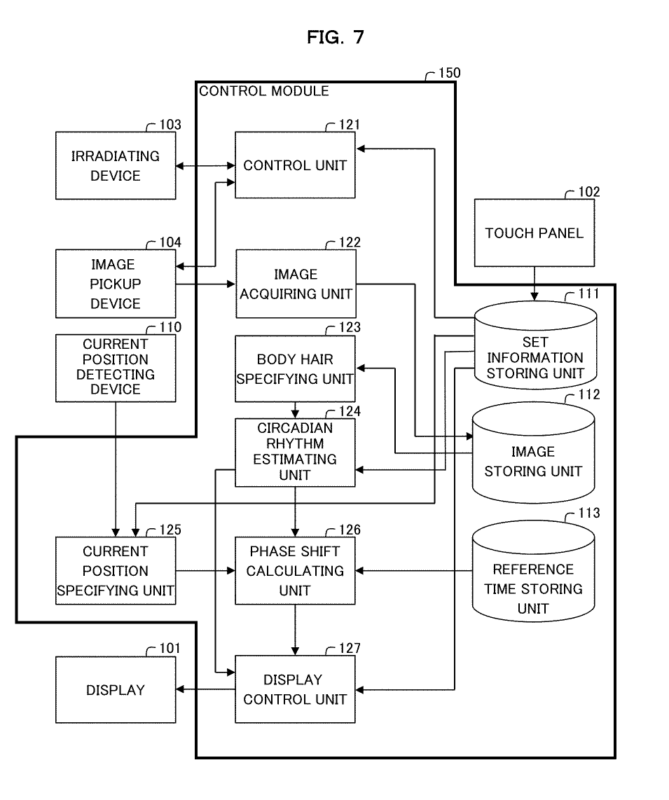

The control module 150 controls each portion of the circadian rhythm examining device 100. For example, the control module 150 controls an irradiation timing of the irradiating device 103, controls an image pickup timing of the image pickup device 104, estimates a circadian rhythm and calculates a phase shift of the circadian rhythm. The details of the function of the control module 150 will be described later with reference to FIG. 7.

The control module 150 can be configured from an IC chip, a microcomputer or the like. for example. In the case in which the control module 150 is configured from the microcomputer, respective function blocks 121 to 127 shown in FIG. 7 include a CPU, an RAM, an ROM and the like and are implemented by an operation of a program stored in a recording medium such as an RAM or an ROM.

(Functional Structure of Control Module 150)

FIG. 7 is a block diagram showing an example of a functional structure of the control module 150 according to the first embodiment of the present invention. As shown in FIG. 7, the control module 150 includes a set information storing unit 111, an image storing unit 112 and a reference time storing unit 113. Moreover, the control module 150 includes, as a functional structure thereof, a control unit 121, an image acquiring unit 122, a body hair specifying unit 123, a circadian rhythm estimating unit 124, a current position specifying unit 125, a phase shift calculating unit 126 and a display control unit 127.

The set information storing unit 111 stores various set information. For example, the set information storing unit 111 stores the following set information. These set information can be set and changed through an operation of the touch panel 102 by a user.

(a) profile information of examination subject (name, age, gender, ID, body height, body weight and the like)

(b) examination place (for example, latitude longitude information, nationality, region, address and the like)

(c) measurement time (for example, every 30 minutes, every hour and the like)

(d) estimation time (for example, after passage of 24 hours, after passage of 48 hours and the like)

(e) attachment portions (for example, hand, leg and the like)

(f) current time to be set to internal clock

(g) current position (for example, latitude • longitude information, nationality, region, address and the like)

(h) setting of image pickup methods (he first to sixth image pickup methods) to be used for examination.

For example, the set information (b) and (g) are used for specifying the current position of the circadian rhythm examining device 100 by the current position specifying unit 125. Moreover, the set information (c) is used when the control unit 121 is to specify a time for picking up the skin image. Furthermore, the set information (d) is used when the circadian rhythm estimating unit 124 is to specify a time for estimating a circadian rhythm. In addition, the set information (h) is used when the control unit 121 is to specify the image pickup method to be used when a skin image is picked up. The set information (a) and (e) are set to the set information storing unit 111 as information to be displayed on the display 101 together with various examination results in the first embodiment.

The reference time storing unit 113 stores a time of a light-dark condition which is predetermined. For example, the reference time storing unit 113 prestores, as the time of the light-dark condition, a time t′ to be set to a time ZT=0 in the light-dark condition as is illustrated in FIG. 2(b). The time of the light-dark condition stored in the reference time storing unit 113 is read by the phase shift calculating unit 126 and is used for calculating the phase shift of the circadian rhythm estimated by the circadian rhythm estimating unit 124. In particular, the reference time storing unit 113 stores times of light-dark conditions every region and time.

The control unit 121 controls the irradiation of light through the irradiating device 103. Moreover, the control unit 121 controls the pickup of the skin image through the image pickup device 104. More specifically, the control unit 121 causes the irradiating device 103 to emit light every time a measurement time (for example, every 30 minutes) set to the set information storing unit 111 arrives. The control unit 121 causes the image pickup device 104 to pickup the skin image when the light emitted from the irradiating device 103 is being irradiated on the skin. Consequently, the image pickup device 104 can pick up the skin image through reflected light of the light irradiated by the irradiating device 103.

As described above, the circadian rhythm examining device 100 includes the irradiating devices 103A to 103F. The control unit 121 selects, from the irradiating devices 103A to 103F, the irradiating device 103 corresponding to the image pickup method (the first to sixth image pickup methods) set to the set information storing unit 111 every time the measurement time arrives, and controls the selected irradiating device 103 to emit light. Consequently, the circadian rhythm examining device 100 can vary the wavelength and vibrating direction of the light to be irradiated on the skin depending on the image pickup method.

For example, in the case of the first image pickup method, the control unit 121 emits white light or broad spectral light from the irradiating device 103A and causes the image pickup device 104 to pick up a skin image through the reflected light. In the case of the second image pickup method, moreover, the control unit 121 emits the white light or the broad spectral light from the irradiating device 103B to cause the image pickup device 104 to pick up a skin image through the reflected light.

In the case of the third image pickup method, furthermore, the control unit 121 first causes the irradiating device 103A to emit the white light or broad spectral light, thereby causing the image pickup device 104 to pick up a skin image (a first image) through reflected light thereof. Next, the control unit 121 causes the irradiating device 103C to emit light having a specific wavelength, thereby causing the image pickup device 104 to pick up a skin image (a second image) through the reflected light.

In the case of the fourth image pickup method, moreover, the control unit 121 first causes the irradiating device 103B to emit the white light or broad spectral light, thereby causing the image pickup device 104 to pick up a skin image (a first image) through reflected light thereof. Next, the control unit 121 causes the irradiating device 103D to emit light having a specific wavelength, thereby causing the image pickup device 104 to pick up a skin image (a second image) through reflected light thereof.

In the case of the fifth image pickup method, furthermore, the control unit 121 causes the irradiating device 103E to emit the white light or broad spectral light, thereby causing the image pickup device 104 to pick up a skin image through reflected light thereof. In the case of the sixth image pickup method, moreover, the control unit 121 causes the irradiating device 103F to emit light having an optional wavelength, thereby causing the image pickup device 104 to pick up a skin image through reflected light thereof.

In the case in which a plurality of image pickup methods is set to the set information storing unit 111, the control unit 121 causes the irradiating device 103 corresponding to each of the image pickup methods to emit light and causes the image pickup device 104 to pick up a skin image through reflected light thereof every image pickup method in such a manner that respective skin images through the image pickup methods can be obtained every time the measurement time arrives. For example, in the case in which the first to sixth image pickup methods are set to the set information storing unit 111, the control unit 121 performs the following controls 1 to 6 in order every time the measurement time arrives.

(Control 1) To cause the irradiating device 103A to emit white light or broad spectral light, thereby causing the image pickup device 104 to pick up a skin image through reflected light thereof.

(Control 2) To cause the irradiating device 103C to emit light having a specific wavelength, thereby causing the image pickup device 104 to pick up a skin image through reflected light thereof.

(Control 3) To cause the irradiating device 103B to emit white light or broad spectral light, thereby causing the image pickup device 104 to pick up a skin image through reflected light thereof.

(Control 4) To cause the irradiating device 103D to emit light having a specific wavelength, thereby causing the image pickup device 104 to pick up a skin image through reflected light thereof.

(Control 5) To cause the irradiating device 103E to emit white light or broad spectral light, thereby causing the image pickup device 104 to pick up a skin image through reflected light thereof.

(Control 6) To cause the irradiating device 103F to emit light having an optional wavelength, thereby causing the image pickup device 104 to pick up a skin image through reflected light thereof.

The image acquiring unit 122 acquires a skin image from the image pickup device 104 every time the image pickup device 104 picks up the skin image. Then, the image acquiring unit 122 stores the skin image acquired from the image pickup deice 104 in the storing unit 112. At this time, the image acquiring unit 122 stores the skin image acquired from the image pickup device 104 in the image storing unit 112 in correspondence to at least an image pickup time of the skin image. The image pickup time of the skin image can be obtained from the internal clock (not shown) of the circadian rhythm examining device 100, for example. In the case in which a plurality of skin images is picked up at a single measurement time (the case of the third or fourth image pickup method and the case in which the image pickup methods are executed), the image acquiring unit 122 acquires these skin images from the image pickup device 104 and stores them in the image storing unit 112.

The image storing unit 112 stores the skin image picked up by the image pickup device 104 and acquired by the image acquiring unit 122. In the case in which a plurality of skin images is picked up at a single measurement time, the image storing unit 112 stores a plurality of images picked up at each measurement time every measurement time. The image storing unit 112 can be configured from a nonvolatile recording element such as a flash memory, for example.

The body hair specifying unit 123 reads the skin images picked up over a plurality of measurement times by the image pickup device 104 from the image storing unit 112, thereby specifying the body hair 20B of the examination subject from the skin image with regard to each of the skin images. In the case in which the skin images are picked up at a single measurement time, the body hair specifying unit 123 reads the images picked up at each measurement time from the image storing unit 112 every measurement time, thereby specifying the body hair 20B of the examination subject from the skin image with regard to each of the skin images every measurement time.

In particular, the body hair specifying unit 123 detects a length of the body hair 20B to be specified from the image with regard to each of the skin images, thereby specifying body hair for a growth period (that is, the body hair 20B having a length varied with time). Characteristics of the skin images to be obtained by the first to sixth image pickup methods are varied. For this reason, the body hair specifying unit 123 specifies the body hair 20B by using a method corresponding to the image pickup method used in the pickup of the skin image.

For example, according to the first or second image pickup methods, it is possible to obtain a skin image in which an image of the skin surface 20A containing a large number of hemoglobin pigments and an image of the body hair 20B containing a large number of melanin pigments have almost equal brightnesses. For this reason, the body hair specifying unit 123 estimates a separation matrix from the skin image to separate a color space vector of melanin and to detect an object containing a large number of melanin pigment components, thereby specifying the object as the body hair 20B by utilizing the conventional known technology, for example.

According to the third and fourth image pickup methods, moreover, there is obtained, as a first image, an image in which an image of the skin surface 20A containing a large number of hemoglobin pigments and an image of the body hair 20B containing a large number of melanin pigments have almost equal brightnesses. Moreover, there is obtained, as a second image, an image in which the image of the skin surface 20A containing a large number of hemoglobin pigments and the image of the body hair 20B containing a large number of melanin pigments have greatly different brightnesses from each other. For this reason, the body hair specifying unit 123 obtains a difference between brightness information of the first image and brightness information of the second image, thereby acquiring a difference image in which the image of the body hair 20B is emphasized. Then, the body hair specifying unit 123 detects an object containing a large number of melanin pigment component from the difference image, thereby specifying the object as the body hair 20B.

According to the fifth image pickup method, moreover, there is obtained a skin image through light reflected from the skin surface 20A (that is, a skin image having a large amount of information related to an uneven state of the skin surface 20A). For this reason, the body hair specifying unit 123 detects an object taking a predetermined contour shape from the skin image, thereby specifying the object as the body hair 20B. In other words, the body hair 20B can be specified irrespective of a color of the skin 20 and a color of the body hair 20B. Therefore, it is possible to specify the body hair 20B from the skin image in any of the case in which a large number of melanin components are contained in the skin 20 and the case in which the body hair 20B contains a small number of melanin components.

According to the sixth image pickup method, moreover, there is obtained a skin image through light reflected from the deep skin layer 20C (that is, a skin image having a large amount of information related to a state of an inner part of the skin). Therefore, the body hair specifying unit 123 detects an object containing a large number of melanin pigment components from the skin image, thereby specifying the object as the body hair 20B present in the deep skin layer 20C.

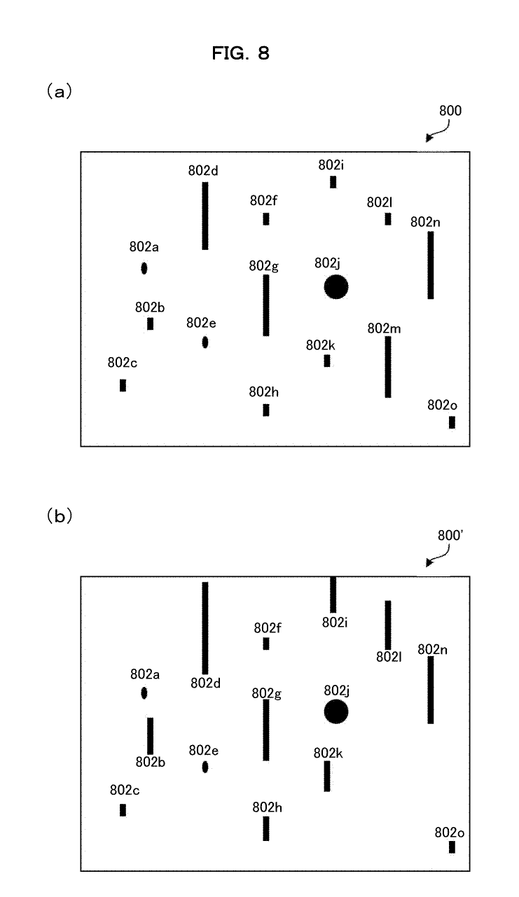

The details of the method of specifying the body hair 20B for the growth period of the examination subject from the skin image will be described later with reference to FIG. 8.

The circadian rhythm estimating unit 124 estimates the circadian rhythm of the examination subject based on a temporal change in a length of the body hair for the growth period which is obtained by specifying the body hair for the growth period from the skin images through the body hair specifying unit 123. The details of the method of estimating the circadian rhythm of the examination subject will be described later with reference to FIG. 2.

The current position specifying unit 125 specifies the current position of the circadian rhythm examining device 100. For example, the current position specifying unit 125 specifies the current position of the circadian rhythm examining device 100 based on a current position signal supplied from the current position detecting device 110. In the case in which the circadian rhythm examining device 100 does not include the current position detecting device 110, the current position specifying unit 125 may specify a current position or an examination place set to the set information storing unit 111 as the current position of the circadian rhythm examining device 100.

The phase shift calculating unit 126 calculates a phase shift of the circadian rhythm estimated by the circadian rhythm estimating unit 124 based on the circadian rhythm estimated by the circadian rhythm estimating unit 124 and the time of the light-dark condition stored in the reference time storing unit 113. In particular, the phase shift calculating unit 126 calculates the phase shift of the circadian rhythm estimated through the circadian rhythm estimating unit 124 by using a time of a light-dark condition corresponding to a current date and time and a current position specified by the current position detecting device 110. The details of the method of calculating the phase shift of a circadian rhythm will be described later with reference to FIG. 2.

The display control unit 127 controls display of various information through the display 101. For example, the display control unit 127 causes the display 101 to display the circadian rhythm of the examination subject estimated by the circadian rhythm estimating unit 124. Moreover, the display control unit 127 causes the display 101 to display the phase shift of the circadian rhythm of the examination subject which is calculated by the phase shift calculating unit 126.

Although the circadian rhythm examining device 100 according to the first embodiment has such a structure as to cause the display 101 to display the circadian rhythm and the phase shift of the circadian rhythm, the present invention is not restricted thereto. For example, the circadian rhythm examining device 100 may be configured to enable export of the circadian rhythm and the phase shift of the circadian rhythm to an external device (for example, a smartphone, a personal computer, a cell phone or the like) through a wireless communication or a cable communication.

[Specific Example of Method of Specifying Body Hair from Skin Image]

Next, a specific example of a method of specifying body hair from a skin image through the circadian rhythm examining device 100 according to the first embodiment will be described with reference to FIG. 8. FIG. 8 is a view showing an example of a skin image picked up by the image pickup device 104 according to the first embodiment of the present invention. A skin image 800 shown in FIG. 8(a) is an example of a skin image picked up by the image pickup device 104 at a first one of measurement times set to the set information storing unit 111. A skin image 800′ shown in FIG. 8(b) is an example of the skin image picked up by the image pickup device 104 at a next measurement time. In particular, the skin images 800 and 800′ are examples of the image on the skin surface picked up by the first or third image pickup method.

In the skin image 800, a plurality of objects 802a to 802o present on the skin surface is projected. In the skin image 800, the objects 802a to 802o include other objects (for example, spots, moles and the like) containing a melanin component in addition to the body hair containing the melanin component. Furthermore, the objects 802a to 802o also include foreign substances (for example, dust or the like) present on the skin surface. The body hair specifying unit 123 specifies, as the body hair for the growth period, any of the objects 802a to 802o which satisfies a predetermined condition.

More specifically, the body hair specifying unit 123 specifies, as the body hair for the growth period, any of the objects 802a to 802o projected onto the skin images 800, 800′ which satisfies all of the following conditions 1 to 3.

(Condition 1) Contain a large number of melanin pigment components.

(Condition 2) Projected onto both the skin image 800 and the skin image 800′ and in the same positions.

(Condition 3) Greater length in the skin image 800′ than the skin image 800.

For instance, in the example shown in FIG. 8, objects 802b, 802d, 802h, 802i, 802k and 802l which satisfy all of the conditions 1 to 3 are specified as the body hair for the growth period by the body hair specifying unit 123. The object information with regard to the objects thus specified as the body hair for the growth period by the body hair specifying unit 123 is stored in a memory of the circadian rhythm examining device 100 or the like. For example, the object information include identification information, position information (for example, XY coordinate values), a length, a difference from a length in a last image, a decision result of a type of the object (that is, the purport of the body hair for the growth period) and the like.

The body hair specifying unit 123 can exclude the following objects from specifying targets serving as the body hair for the growth period by specifying, as the body hair for the growth period, the object which satisfies all of the conditions 1 to 3.

Objects other than the body hair which are formed on the skin surface and contain a large number of melanin pigment components, for example, spots, moles and the like (because they do not satisfy the condition 3).

Any of foreign substances present on the skin surface which does not contain a large number of melanin pigment components, for example, dust (because they do not satisfy the conditions 1 and 3).

Any of foreign substances present on the skin surface which contains a large number of melanin pigment components, for example, fallen hair and the like (because they do not satisfy the condition 3).

Body hair for a non-growth period (because they do not satisfy the condition 3).

It is preferable that the body hair specifying unit 123 should specify the body hair for the growth period by using the following condition 1′ in place of the condition 1 in the case in which the body hair for the growth period is to be specified from the skin image picked up by the fifth image pickup method. The reason is that there is obtained a skin image in which a contour of body hair on a skin surface is emphasized by the fifth image pickup method.

(Condition 1′) To have a size width within a predetermined range (for example, in a range of 30 to 150 μm) and to have a size in which a length is greater than a width (that is, a vertically long shape).

Moreover, the body hair specifying unit 123 may store, in the memory of the circadian rhythm examining device 100 or the like, object information about an object which is not specified as the body hair for the growth period in the same manner as the object specified as the body hair for the growth period. In this case, the object information includes the decision result of the type of the object (that is, information indicating whether the object is the body hair for the growth period or not) and the like in addition to identification information, position information (for example, XY coordinate values) and a length, for example.

In the case in which the object which is not specified as the body hair for the growth period is thus stored in the memory, the body hair specifying unit 123 may manage a position, a shape, a size and the like of the object, thereby using the object as a reference point for performing alignment with the skin image 800 and the skin image 800′.

Referring to the skin image 800 and the skin image 800′ shown in FIG. 8, for easy understanding of explanation, a skin is shown in a white color and objects present on the skin surface are shown in a black color. However, the colors of the skin and the body hair projected actually onto the skin image are varied depending on the image pickup methods (the first to sixth image pickup methods).

For example, in the case in which the skin image is picked up by the first or second image pickup method, there is obtained a skin image in which the skin becomes reddish tinge by a hemoglobin pigment component in an inner part of the skin and body hair generally has a black color due to a melanin pigment component. In the case in which the skin image is picked up by the third or fourth image pickup method, moreover, an intensity of reflected light of the melanin component with respect to the hemoglobin component is relatively increased. As compared with the skin image to be picked up by the first or second image pickup method, therefore, there is obtained a skin image in which a reddish tinge of the skin is weakened, while the black color of the body hair is emphasized.

In the case in which the skin image is picked up by the fifth image pickup method, furthermore, there is obtained a skin image having color information about the inner part of the skin (a reddish tinge generated by the hemoglobin pigment component or the like) removed and a large amount of information related to an uneven state of the skin surface. In other words, there is obtained a skin image in which a contour of each object present on a skin surface is emphasized. In the skin image to be obtained by the fifth image pickup method, an object beneath an epidermis is not recognized. Therefore, only an object present on the epidermis (for example, body hair, dust or the like) is basically projected.

In the case in which a skin image is picked up by the sixth image pickup method, moreover, there is obtained a skin image from which light reflected from a skin surface is removed and which has a large amount of information about the inner part of a skin. As compared with the skin image to be picked up by the first image pickup method, therefore, there is obtained a skin image in which body hair in the inner part of the skin is projected and a reddish tinge of the skin is emphasized.

In the case in which a plurality of skin images is picked up by a plurality of image pickup methods for a single measurement period, the body hair specifying unit 123 specifies the body hair from the skin image as described above every image pickup method. For example, in the case in which a skin image through the light reflected from the skin surface (by any of the first, third and fifth image pickup methods) and a skin image through light reflected from a deep skin layer (by any of the second, fourth and sixth image pickup methods) are picked up at a single measurement time, the body hair specifying unit 123 performs a processing for specifying body hair as described above for each of the skin image through the light reflected from the skin surface and the skin image through the light reflected from the deep skin layer.

In this case, the circadian rhythm examining device 100 may separately mange the object information about the body hair specified from the image through the light reflected from the skin surface and the object information about the body hair specified from the image through the light reflected from the deep skin layer. In this case, the circadian rhythm estimating unit 124 may estimate a circadian rhythm from the object information about the body hair specified from the image through the light reflected from the skin surface, and furthermore, may estimate a circadian rhythm from the object information about the body hair specified based on the image through the light reflected from the deep skin layer.

[Specific Example of Method of Estimating Circadian Rhythm and Method of Calculating Phase Shift of Circadian Rhythm]

Next, a specific example of a method of estimating a circadian rhythm and a method of calculating a phase shift of a circadian rhythm by the circadian rhythm examining device 100 according to the first embodiment will be described with reference to FIG. 2 again. For example, the circadian rhythm examining device 100 estimates a circadian rhythm of an examination subject in accordance with the following procedure of (1) to (2). Then, the circadian rhythm examining device 100 calculates the phase shift of the circadian rhythm in accordance with the following procedure of (3) to (5).

(1) The circadian rhythm estimating unit 124 obtains a temporal change in the length of the body hair for the growth period shown in FIG. 2(a) based on the length of the body hair for the growth period which is specified by the body hair specifying unit 123 from each of the skin images picked up by the image pickup device 104 over a plurality of measurement times. In the case in which the body hairs for the growth period are specified from the skin images by the body hair specifying unit 123, the temporal change in the length of the body hair is obtained by using an average value of the lengths of the body hair. It is preferable that the skin images should be picked up for 24 hours or more and it is more preferable that the skin images should be picked up at a suitable interval for observing the growth of the body hair (every 30 minutes, for example).

(2) The circadian rhythm estimating unit 124 differentiates the temporal change in the body hair obtained in the (1) to acquire a temporal change in a growth speed of the body hair shown in FIG. 2(b), thereby estimating the temporal change as a circadian rhythm of an examination subject.

(3) The phase shift calculating unit 126 sets a time t that a peak in the circadian rhythm estimated in the (2) (that is, a peak of a growth speed of the body hair) is reached as the interior time CT=0 of the examination subject for convenience as shown in FIG. 2(b). By future studies or the like, however, there is also a possibility that the knowledge of proper setting of the interior time CT=0 to a time shifted from the time t to reach the peak in the circadian rhythm might be obtained. In this case, it is also possible to change the processing to be performed by the phase shift calculating unit 126 in order to set the interior time CT=0 to the time shifted from the time t.

(4) The phase shift calculating unit 126 specifies the time t′ that the time ZT=0 in the light-dark condition which is the time of the light-dark condition is set by referring to the reference time storing unit 113. Herein, the phase shift calculating unit 126 specifies the time t′ corresponding to a current date and a current position. Consequently, the phase shift calculating unit 126 can use the time ZT in a proper light-dark condition corresponding to a current date and a current position to calculate the phase shift of the circadian rhythm depending on a fluctuation in the time ZT in the light-dark condition based on a sunrise time corresponding to a region and a time. Accordingly, it is possible to enhance calculation precision in a phase shift of the circadian rhythm. The current date can be obtained from the internal clock provided in the circadian rhythm examining device 100, for example. Moreover, the current position can be specified by the current position specifying unit 125.

(5) The phase shift calculating unit 126 calculates, as a phase shift for a time in a light-dark condition of the circadian rhythm estimated in the (2), a time difference of the time t that the interior time CT=0 is set for the time t′ that the time ZT=0 in the light-dark condition is set. As a result, if the interior time CT is almost equal to the time ZT in the light-dark condition, it is decided that there is no separation between an interior time and an external environment.

If the interior time CT is earlier than the time ZT in the light-dark condition, it is decided that the interior time CT advances with respect to the external environment. In a typical example, the following result is obtained for a morning person. If the interior time CT is later than the time ZT in the light-dark condition, moreover, it is decided that the interior time CT is delayed with respect to the external environment. In a typical example, the following result is obtained for a night person.

The circadian rhythm examining device 100 can cause a user (an examination subject) to grasp the circadian rhythm and the phase shift of the circadian rhythm thus obtained by causing the display 101 to display them as shown in FIG. 10(a), for example. In addition, the circadian rhythm examining device 100 may output the circadian rhythm and the phase shift of the circadian rhythm to an external apparatus, for example. In this case, the circadian rhythm and the phase shift of the circadian rhythm output to the external apparatus can be utilized for diagnosis to be performed by a doctor or the like, for example.

It is possible to decide whether the circadian rhythm of the examination subject has a 24-hour cycle or not depending on the circadian rhythm obtained in the (2). For example, in the case in which a peak in the circadian rhythm obtained in the (2) (that is, a peak of a growth speed of body hair) has no interval of 24 hours, it is possible to decide that the circadian rhythm of the examination subject does not have the 24-hour cycle. In other words, it is decided that the examination subject is of a non-24-hour type (non-24). For example, the following result is obtained for a patient falling ill, for example, having jet lag or other circadian rhythm sleep disorders or the like.

Herein, the growth speed of the body hair is varied or a change pattern of the growth speed of the body hair is varied depending on a profile of the examination subject (for example, an age, a gender or the like) and an attachment portion. For example, the growth speed differs for upper limbs, lower limbs or the like and the change pattern of the growth speed has a slight difference. Therefore, the circadian rhythm examining device 100 may prestore, in a memory or the like, a correction value corresponding to the profile of the examination subject (for example, an age, a gender or the like) and the attachment portion and the circadian rhythm estimating unit 124 may correct (that is, may adjust the time before or after the time t) a time of the interior time CT=0 from the time t to be the peak in the circadian rhythm based on the correction value corresponding to the profile of the examination subject (for example, an age, a gender or the like) and the attachment portion. Consequently, the circadian rhythm examining device 100 can enhance estimation precision in the circadian rhythm to be estimated by the circadian rhythm estimating unit 124 and precision in the phase shift of the circadian rhythm to be calculated by the phase shift calculating unit 126.

[Example of Processing to be Performed by Circadian Rhythm Examining Device 100]

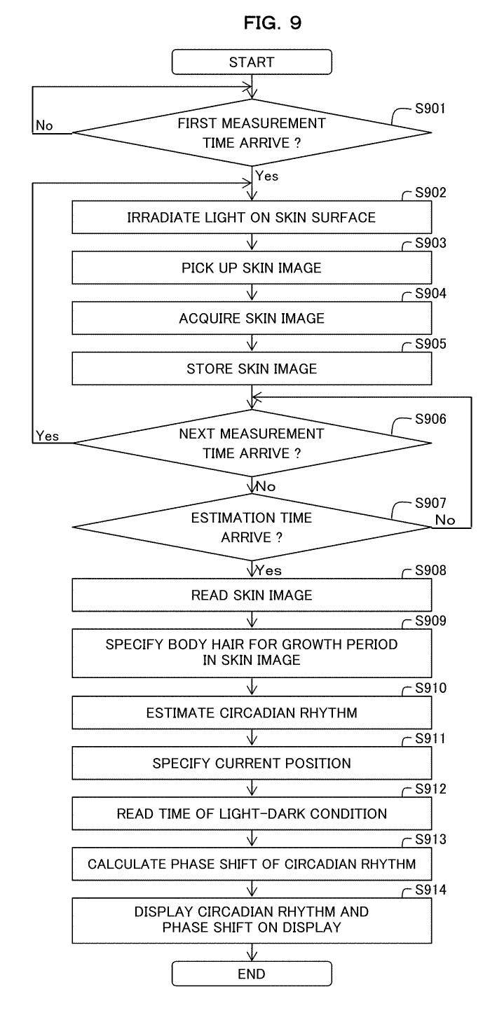

FIG. 9 is a flow chart showing an example of a processing to be performed by the circadian rhythm examining device 100 according to the first embodiment of the present invention. The processing shown in FIG. 9 is started to be executed when a predetermined event for starting the examination of the circadian rhythm of the examination subject in the circadian rhythm examining device 100 is generated (for example, the examination subject performs an operation for starting the examination of the circadian rhythm over the circadian rhythm examining device 100) in a state in which the circadian rhythm examining device 100 is attached to the examination subject, for example.

First of all, the control unit 121 decides whether a first measurement time set to the set information storing unit 111 arrives or not (Step S901). If the control unit 121 decides that the first measurement time arrives (Step S901: Yes), the circadian rhythm examining device 100 advances the processing to Step S902. On the other hand, if the control unit 121 decides that the first measurement time does not arrive (Step S901: No), the circadian rhythm examining device 100 executes the processing of Step S901 again.

In Step S902, the irradiating device 103 irradiates light on the skin of the examination subject by the control of the control unit 121. In that state, the image pickup device 104 picks up a skin image by the control of the control unit 121 (Step S903). Then, the image acquiring unit 122 acquires the skin image picked up in Step S903 (Step S904). Furthermore, the image acquiring unit 122 stores, in the image storing unit 112, the skin image acquired in Step S904 (Step S905).

Thereafter, the control unit 121 decides whether a next measurement time set to the set information storing unit 111 arrives or not (Step S906). Herein, if the control unit 121 decides that the next measurement time arrives (Step S906: Yes), the circadian rhythm examining device 100 executes a processing after Step S902 again. Thus, the processing of Step S902 to S905 is executed every predetermined measurement times. Consequently, a plurality of skin images picked up by the image pickup device 104 is stored in the image storing unit 112. On the other hand, if the control unit 121 decides that the next measurement time does not arrive (Step S906: No), the circadian rhythm examining device 100 advances the processing to Step S907.

In Step S907, the circadian rhythm estimating unit 124 decides whether an estimation time for estimating the circadian rhythm of the examination subject arrives or not. Herein, if the circadian rhythm estimating unit 124 decides that the estimation time for estimating the circadian rhythm of the examination subject arrives (Step S907: Yes), the circadian rhythm examining device 100 advances the processing to Step S908. On the other hand, if the circadian rhythm estimating unit 124 decides that the estimation time for estimating the circadian rhythm of the examination subject does not arrive (Step S907: No), the circadian rhythm examining device 100 executes the processing after Step S906 again.

In Step S908, the circadian rhythm estimating unit 124 reads, from the image storing unit 112, a plurality of skin images stored in the image storing unit 112 in Step S905. Then, the body hair specifying unit 123 specifies body hair for a growth period in the skin image for each of the skin images read in Step S908 (Step S909). Furthermore, the circadian rhythm estimating unit 124 estimates the circadian rhythm of the examination subject based on a temporal change in the length of the body hair for the growth period which is obtained by specifying the body hair for the growth period from the skin images in Step S909 (Step S910).

Thereafter, the current position specifying unit 125 specifies a current position of the examination subject (Step S911). Then, the phase shift calculating unit 126 reads, from the reference time storing unit 113, a time of a light-dark condition corresponding to a current position of the examination subject specified in Step S911 and a current date and time (Step S912). Furthermore, the phase shift calculating unit 126 calculates the phase shift of the circadian rhythm of the examination subject based on the time of the light-dark condition read in step S912 and the circadian rhythm of the examination subject estimated in Step S910 (Step S913).

Subsequently, the display control unit 127 causes the display 101 to display the circadian rhythm of the examination subject estimated in Step S910 and the phase shift of the circadian rhythm of the examination subject calculated in Step S913 (Step S914) and the circadian rhythm examining device 100 ends the processing shown in FIG. 9.

In the first embodiment, a plurality of skin images is acquired at a single measurement time in some cases. In the case in which the third or fourth image pickup method are applied, for example, it is necessary to acquire a skin image through white light and a skin image through light having a specific wavelength at a single measurement time respectively. In the case in which at least two of the first to sixth image pickup methods are applied in combination, moreover, it is necessary to acquire the skin image corresponding to the image pickup method every image pickup method at a single measurement time. In these cases, the circadian rhythm examining device 100 acquires a plurality of skin images at a single measurement time by repetitively executing Steps S902 to S905 while changing the type of light to be irradiated on the skin by the control of the control unit 121.

Although the circadian rhythm and the phase shift of the circadian rhythm are obtained, and then, the circadian rhythm and the phase shift of the circadian rhythm are immediately displayed on the display 101 in the processing shown in FIG. 9, moreover, the present invention is not restricted thereto. For example, it is also possible to store the circadian rhythm and the phase shift of the circadian rhythm in the memory of the circadian rhythm examining device 100 and to read the circadian rhythm and the phase shift of the circadian rhythm from the memory in an optional timing (for example, in the case in which a user performs an operation for causing the display 101 to display the circadian rhythm and the phase shift of the circadian rhythm for the circadian rhythm examining device 100), thereby causing the display 101 to display them.

[Example of Display Screen]

FIG. 10 is a view showing an example of a display screen to be displayed by the circadian rhythm examining device 100 (the display 101) according to the first embodiment of the present invention.

A screen 610 shown in FIG. 10(a) is an example of the display screen for displaying information about examination of a circadian rhythm. The screen 610 includes four types of information 611 to 614. In an example of FIG. 10(a), the information 611 includes a current date and time, a current place and a remaining battery. Moreover, the information 612 includes the interior time CT based on the circadian rhythm estimated by the circadian rhythm estimating unit 124, the time ZT in the light-dark condition, and the phase shift of the circadian rhythm calculated by the phase shift calculating unit 126. In an example shown in FIG. 10(a), the interior time CT is delayed by four minutes from the time ZT in the light-dark condition. Moreover, the information 613 includes a graphic representing a pattern of the circadian rhythm estimated by the circadian rhythm estimating unit 124. Furthermore, the information 614 includes an attachment portion of the circadian rhythm examining device 100 and an ID, a name, a gender and an age as profile information of a user (an examination subject).

A screen 620 shown in FIG. 10(b) is an example of a menu screen for giving access to various functions of the circadian rhythm examining device 100. The screen 620 includes information 621 and four buttons 622 to 625. The information 621 includes a current date and time, a current place and a remaining battery in the same manner as the information 611.

The button 622 indicated as “Regist Patient” serves to display an input screen of a profile (for example, a name, an age, a gender, an ID, a body height, a body weight and the like) of the user (the examination subject). The button 623 indicated as “Preview Data” serves to cause the display 101 to preview display the skin image picked up by the image pickup device 104, various examination results and the like.

The button 624 indicated as “Settings” serves to cause the display 101 to display a setting screen for performing various settings to the circadian rhythm examining device 100 (for example, a current time, a current position, an image pickup method to be used for examination and the like). The button 625 indicated as “Export” serves to cause the display 101 to display a screen for outputting various information (for example, a skin image stored in the image storing unit 112, various examination results and the like) to an external apparatus.

As described above, according to the circadian rhythm examining device 100 in accordance with the first embodiment, it is possible to estimate the circadian rhythm of the examination subject from the skin images obtained by simply picking up the skin image plural times without requiring gene measurement, cell culture, frequent blood collection, analysis in special research facilities and the like. The phase shift of the circadian rhythm of the examination subject can be calculated based on the estimated circadian rhythm. According to the circadian rhythm examining device 100 in accordance with the first embodiment, therefore, it is possible to easily grasp the circadian rhythm of the examination subject and the phase shift thereof. According to the circadian rhythm examining device 100 in accordance with the first embodiment, particularly, it is possible to calculate the phase shift of the circadian rhythm with high precision based on the time of the light-dark condition corresponding to the current position and the current date and time.

According to the circadian rhythm examining device 100 in accordance with the first embodiment, moreover, the irradiating device 103 and the image pickup device 104 are configured in such a manner that various skin images can be picked up by using plural types of light having different wavelengths and vibrating directions. According to the circadian rhythm examining device 100 in accordance with the first embodiment, therefore, it is possible to specify body hair in various states which is present on the skin of the examination subject with high precision. For example, it is possible to specify body hair containing a large number of melanin components by the first or second image pickup method. By the third or fourth image pickup method, furthermore, an image having a melanin pigment component emphasized is obtained. Therefore, it is possible to specify the body hair containing a large number of melanin components with higher precision.

By the fifth image pickup method, moreover, there is obtained a skin image including a large amount of information about an uneven state of a skin surface. In both the case in which a content of the melanin pigment components in the skin is large and the case in which the content of the melanin pigment component in the body hair is small (for example, in the case of white hair), therefore, it is possible to easily specify the body hair. By the sixth image pickup method, moreover, it is possible to pick up an image having a large amount of information about a deep skin layer. Therefore, it is possible to specify the body hair present in the skin. By executing the image pickup methods in combination, furthermore, it is possible to further enhance specific precision in the body hair.

According to the circadian rhythm examining device 100 according to the first embodiment, moreover, it is possible to execute all examining steps (that is, from the pickup of the skin image to the display of various examination results on the display 101) shown in FIG. 9 in a state in which the circadian rhythm examining device 100 is maintained to be fixed to the skin surface of the examination subject. According to the circadian rhythm examining device 100 in accordance with the first embodiment, therefore, it is possible to perform measurement over the same measuring position for a long period of time without requiring a great deal of time and labor for attachment/removal to/from the examination subject and an operation or execution of alignment of the examining device every image pickup period.

### Second Embodiment

[Example of Structure of Circadian Rhythm Examining System 10]

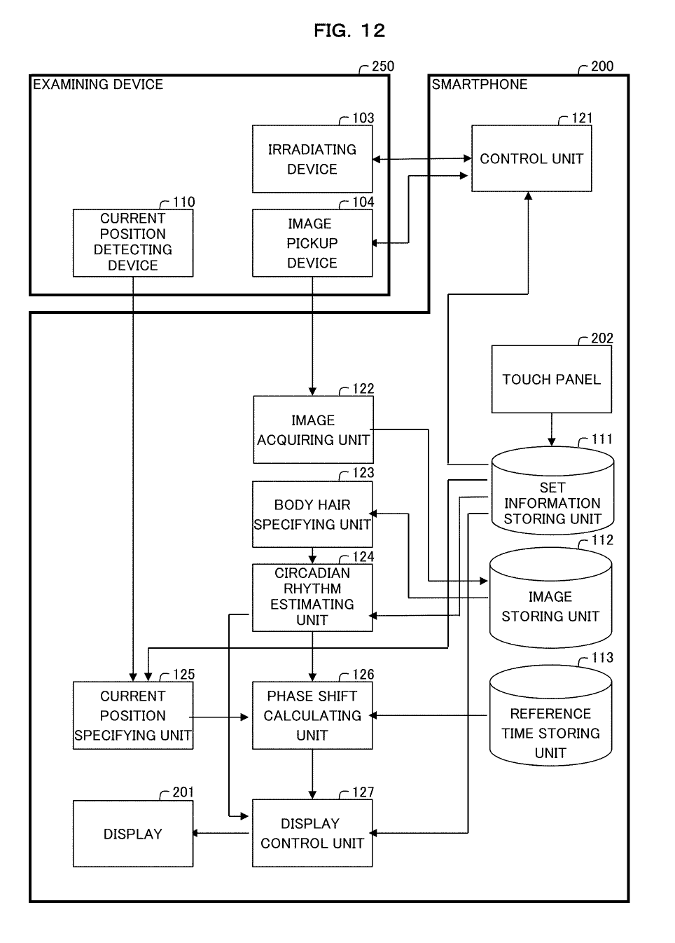

A second embodiment according to the present invention will be described below with reference to FIGS. 11 and 12. Description will be given to changes from the first embodiment with regard to the second embodiment. FIG. 11 is a view showing an example of a structure of a circadian rhythm examining system 10 according to the second embodiment of the present invention. FIG. 12 is a block diagram showing an example of a functional structure of the circadian rhythm examining system 10 according to the second embodiment of the present invention.

The circadian rhythm examining system 10 shown in FIG. 11 includes a skin image pickup device 250 and a smartphone 200. The skin image pickup device 250 is attached to a skin surface of an examination subject in the same manner as the circadian rhythm examining device 100 according to the first embodiment and serves to pick up the skin image of the examination subject. The smartphone 200 is a portable information terminal possessed by the examination subject. The smartphone 200 is an example of an information processing device described in claims. The skin image pickup device 250 and the smartphone 200 can make a communication with each other through a wireless communication (for example, Wi-Fi, Bluetooth (registered trademark) or the like) or a cable communication (for example, a USB cable or the like).

the device structure of the skin image pickup device 250 is different from the device structure of the circadian rhythm examining device 100 shown in FIG. 3 in that the display 101, the touch panel 102 and the control module 150 are not provided.

[Functional Structure of Circadian Rhythm Examining System 10]

The circadian rhythm examining system 10 having this structure is configured to implement the function of the circadian rhythm examining device 100 according to the first embodiment by the skin image pickup device 250 and the smartphone 200.

For example, as shown in FIG. 12, the irradiating device 103, the image pickup device 104 and the current position detecting device 110 in the components of the circadian rhythm examining device 100 according to the first embodiment (see FIG. 7) are provided in the skin image pickup device 250 according to the second embodiment. On the other hand, the components of the control module 150 (the functional blocks 121 to 127, the set information storing unit 111, the image storing unit 112 and the reference time storing unit 113) in the components of the circadian rhythm examining device 100 according to the first embodiment (see FIG. 7) are provided in the smartphone 200 according to the second embodiment.

Although the display 101 and the touch panel 102 in the circadian rhythm examining device 100 are used as a display device and an input device in the first embodiment, moreover, a display 201 and a touch panel 202 in the smartphone 200 is used in the second embodiment.

In the second embodiment, a function in which attachment to the skin surface of the examination subject is essential (irradiation of light on a skin, pickup of a skin image, detection of a current position) is implemented by the skin image pickup device 250 and the other functions are implemented by the smartphone 200 (an existing information processing device). In the second embodiment, therefore, the skin image pickup device 250 can have a minimum structure and a cost related to the image pickup device 250 can be reduced.

In other words, according to the second embodiment, it is possible to construct the circadian rhythm examining system 10 capable of examining the same circadian rhythm as that of the first embodiment without requiring other special devices and applications by simply preparing the skin image pickup device 250 having a communicating function with the smartphone 200 and introducing an application for implementing the function shown in FIG. 12 into the smartphone 200.

Although the smartphone 200 is used as an example of the information processing device described in the claims in the second embodiment, the present invention is not restricted thereto. For example, another information processing device such as a personal computer or a cell phone may be used as the information processing device described in the claims.

Although the display 201 and the touch panel 202 in the smartphone 200 are used as a display device and an input device in the second embodiment, moreover, the present invention is not restricted thereto. For example, the display and the touch panel in the skin image pickup device 250 may be used as the display device and the input device.

Although the current position of the skin image pickup device 250 is detected by the current position detecting device 110 of the skin image pickup device 250 in the second embodiment, moreover, the present invention is not restricted thereto. For example, the current position of the smartphone 200 may be detected by the current position detecting device (for example, a GPS) of the smartphone 200. The reason is that the current position indicated by the skin image pickup device 250 and the current position indicated by the smartphone 200 are the same positions because the skin image pickup device 250 and the smartphone 200 are possessed by the same examination subject.

### Third Embodiment

[Example of Structure of Circadian Rhythm Examining System 10′]

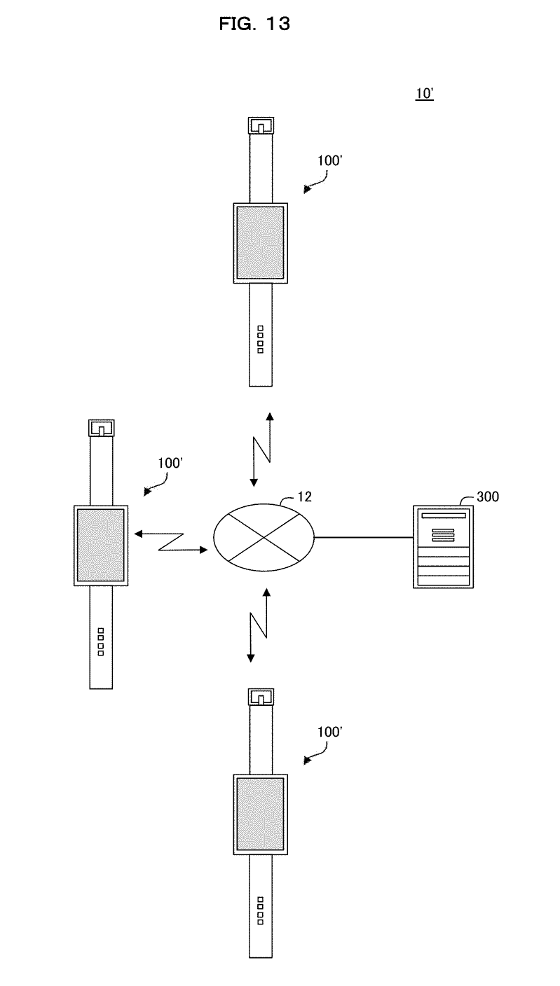

A third embodiment according to the present invention will be described below with reference to the drawings. FIG. 13 is a view showing an example of a structure of a circadian rhythm examining system 10′ according to the third embodiment of the present invention. The circadian rhythm examining system 10′ includes a plurality of circadian rhythm examining devices 100′ and a server 300.

Each of the circadian rhythm examining devices 100′ is attached to the skin surface of the examination subject to examine the circadian rhythm of the examination subject in the same manner as the circadian rhythm examining device 100 according to the first embodiment. Each circadian rhythm examining device 100′ can be connected to a communication network 12 (for example, a wireless LAN, a cell phone network, INTERNET or the like) via a predetermined wireless communication method (for example, Wi-Fi, Bluetooth (registered trademark) or the like) or a cable. Consequently, each circadian rhythm examining device 100′ can give access to the server 300 through the communication network 12.

[Functional Structure of Circadian Rhythm Examining System 10′]

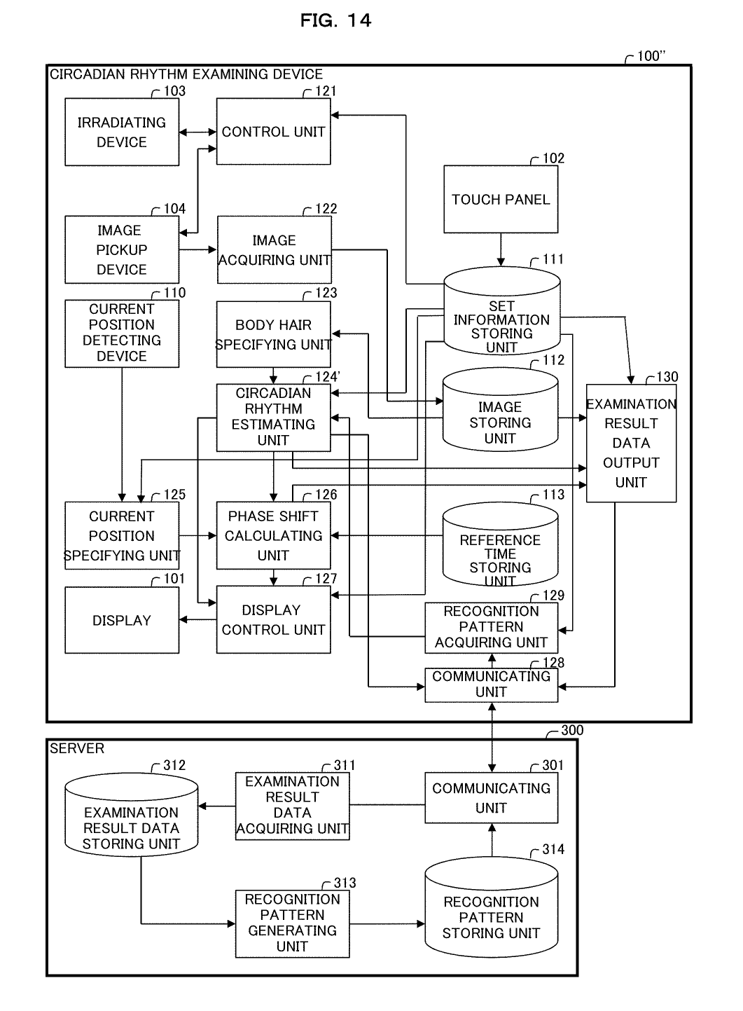

FIG. 14 is a block diagram showing an example of a functional structure of the circadian rhythm examining system 10′ according to the third embodiment of the present invention. The circadian rhythm examining device 100′ according to the third embodiment shown in FIG. 14 is different from the circadian rhythm examining device 100 according to the first embodiment shown in FIG. 7 in that a communicating unit 128, a recognition pattern acquiring unit 129 and an examination result data output unit 130 are further provided and that a circadian rhythm estimating unit 124′ is provided in place of the circadian rhythm estimating unit 124.

The communicating unit 128 controls a communication with the server 300 through the communication network 12. The recognition pattern acquiring unit 129 acquires a recognition pattern of a circadian rhythm from the server 300 through the communicating unit 128. In particular, the recognition pattern acquiring unit 129 specifies a profile and an attachment portion of an examination subject by referring to the set information storing unit 111 and acquires, from the server 300, the recognition pattern of the circadian rhythm corresponding to the profile and the attachment portion of the examination subject which are specified.

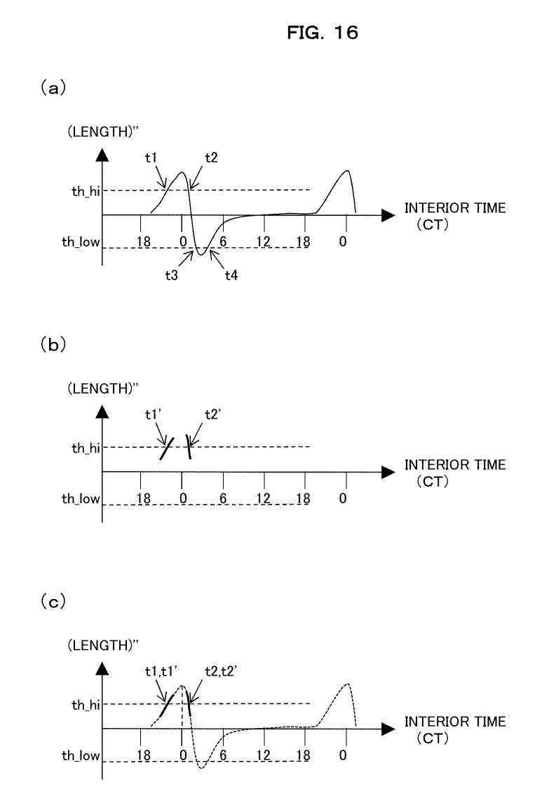

The recognition pattern of the circadian rhythm acquired by the recognition pattern acquiring unit 129 is supplied to a circadian rhythm estimating unit 124′. The circadian rhythm estimating unit 124′ has a function for estimating a circadian rhythm (a second estimating function) by a method using the recognition pattern of the circadian rhythm in addition to a function for estimating a circadian rhythm (a first estimating function) by the method described in the first embodiment. The details of the second estimating function for estimating a circadian rhythm by using the recognition pattern of the circadian rhythm will be described later with reference to FIG. 16.

The examination result data output unit 130 outputs examination result data to the server 300 through the communicating unit 128. The examination result data include a skin image picked up by the image pickup device 104, a circadian rhythm estimated by the circadian rhythm estimating unit 124′, a phase shift calculated by the phase shift calculating unit 126, profile information of the examination subject which are stored in the set information storing unit 111 (an age, a gender, an ID, a body height, a body weight and the like), set information about an attachment portion stored in the set information storing unit 111 and the like.

On the other hand, the server 300 includes a communicating unit 301, an examination result data acquiring unit 311, an examination result data storing unit 312, a recognition pattern generating unit 313 and a recognition pattern storing unit 314. The communicating unit 301 controls a communication with the circadian rhythm examining device 100′ through the communication network 12.

The examination result data acquiring unit 311 acquires examination result data from each of the circadian rhythm examining devices 100′ through the communicating unit 301. The examination result data storing unit 312 stores the examination result data of the circadian rhythm examining devices 100′ which are acquired by the examination result data acquiring unit 311. The examination result data to be acquired by the examination result data acquiring unit 311 includes the circadian rhythm of the examination subject. Accordingly, the examination result data acquiring unit 311 functions as a circadian rhythm acquiring unit described in claims.

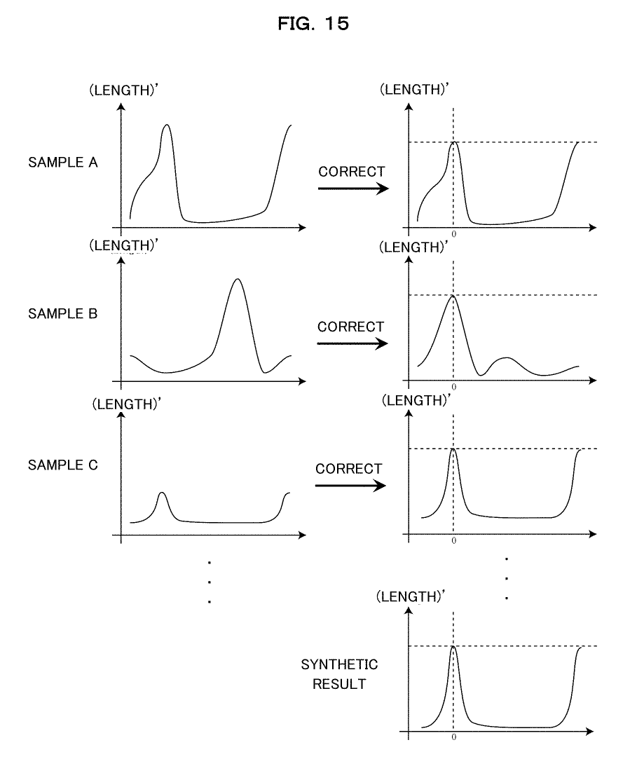

The recognition pattern generating unit 313 corrects and synthesizes a plurality of circadian rhythm waveforms stored in the examination result data storing unit 312 (excluding a circadian rhythm estimated by a method using a recognition pattern which will be described later (see FIG. 16)), thereby generating the recognition pattern of the circadian rhythm. The recognition pattern storing unit 314 stores the recognition pattern of the circadian rhythm generated by the recognition pattern generating unit 313. The details of the method of generating the recognition pattern of the circadian rhythm by synthesis of the circadian rhythm waveforms will be described with reference to FIG. 15.

In particular, the recognition pattern generating unit 313 synthesizes the waveforms of the circadian rythms every profile item (for example, an age, a gender or the like) and attachment portion, thereby generating a recognition pattern of the circadian rhythm every profile item and attachment portion. Correspondingly, the recognition pattern storing unit 314 stores the recognition patterns of the circadian rhythms every profile item and attachment portion.

The recognition pattern of the circadian rhythm stored in the recognition pattern storing unit 314 is transmitted to the circadian rhythm examining device 100′ properly (for example, periodically or when a request is received from the circadian rhythm examining device 100′) through the communicating unit 301. In the circadian rhythm examining device 100′, the recognition pattern acquiring unit 129 acquires the recognition pattern of the circadian rhythm through the communicating unit 128. Consequently, the circadian rhythm estimating unit 124′ can estimate the circadian rhythm by using the recognition pattern of the circadian rhythm.

(Generation of Recognition Pattern of Circadian Rhythm)

FIG. 15 is a chart showing an example of synthesis of circadian rhythm waveforms through the server 300 according to the third embodiment of the present invention. The recognition pattern generating unit 313 of the server 300 corrects and synthesizes waveforms of the circadian rhythms (waveforms of a temporal change in a growth speed of body hair) acquired from the circadian rhythm examining devices 100′, thereby generating the recognition patterns of the circadian rhythms.

For example, the recognition pattern generating unit 313 corrects and synthesizes the waveforms of the circadian rhythms obtained from the circadian rhythm examining devices 100′, thereby generating the recognition patterns of the circadian rhythms as shown in FIG. 15. At this time, the recognition pattern generating unit 313 multiplies each of the waveforms of the circadian rhythms by such a coefficient as to align heights of peaks (that is, peaks of growth speeds of the body hair) with each other, thereby synthesizing the waveforms of the circadian rhythms as shown in FIG. 15 in order to prevent the waveforms of the circadian rhythms of people having extremely high or low growth speeds from being synthesized exactly. Moreover, the recognition pattern generating unit 313 aligns peak positions (positions in a time base direction) of the circadian rhythms with each other, thereby synthesizing the waveforms of the circadian rhythms as shown in FIG. 15 based on the fact that an internal clock of each person has an individual difference.

Consequently, the recognition pattern generating unit 313 generates such a recognition pattern as to enable estimation of a circadian rhythm from a waveform of a growth speed of body hair obtained by an examination for less than 24 hours even if the examination is not continuously performed with the circadian rhythm examining device 100′ attached to a body for 24 hours or more.

The recognition pattern generating unit 313 excludes, from a synthesis target, the circadian rhythm estimated by a method using a recognition pattern which will be described later (see FIG. 16). Such a circadian rhythm has the same waveform as an existing recognition pattern and is not proper for being a specimen in the generation of a new recognition pattern.

The circadian rhythm examining system 10′ according to the third embodiment repetitively performs the above-mentioned circadian rhythm recognition pattern generation processing properly (for example, periodically, in a timing designated by a manager or the like), thereby enabling convergence of the recognition pattern of the circadian rhythm on a standard circadian rhythm gradually. For example, the standard pattern acquiring unit 129 of the circadian rhythm examining device 100′ can acquire the newest circadian rhythm recognition pattern from the server 300 through the communicating unit 128 properly (for example, periodically, in a timing designated by a user, or the like). Consequently, the circadian rhythm estimating unit 124′ of the circadian rhythm examining device 100′ can estimate the circadian rhythm from the waveform of the growth speed of the body hair obtained by the examination for less than 24 hours by using the newest circadian rhythm recognition pattern acquired from the server 300.

(Method of Estimating Circadian Rhythm Using Recognition Pattern of Circadian Rhythm)

FIG. 16 is a chart showing an example of a method of estimating a circadian rhythm using a recognition pattern according to the third embodiment of the present invention. As described above, the circadian rhythm estimating unit 124′ can estimate the circadian rhythm from the waveform of the growth speed of the body hair which is obtained by an examination for less than 24 hours by using the recognition pattern of the circadian rhythm acquired from the server 300.

More specifically, if waveforms for a part of measured values (the growth speed of the body hair) are obtained before passage of 24 hours, the circadian rhythm estimating unit 124′ can estimate a circadian rhythm of an examination subject from the recognition pattern by collating the waveforms of the part of measured values with the waveform of the recognition pattern of the circadian rhythm acquired from the server 300.

For example, FIG. 16(a) shows an example of the recognition pattern of the circadian rhythm. As shown in FIG. 16(a), a variation in the growth speed of the body hair reaches a predetermined upper limit reference value (th-hi in the drawing) only twice (t1, t2 in the drawing) for 24 hours. Moreover, the variation in the growth speed of the body hair reaches a predetermined lower limit reference value (th-low in the drawing) only twice (t3, t4 in the drawing) for 24 hours.

Even if the measurement of the body hair is not performed for 24 hours or more, therefore, the circadian rhythm estimating unit 124′ partially generates a graph representing the variation in the growth speed of the body hair from a part of measurement results and specifies two times t1′ and t2′ (or t3′, t4′) that the predetermined upper limit reference value (or the lower limit reference value) is reached from the partial graph as shown in FIG. 16(b).

As shown in FIG. 16(c), the circadian rhythm estimating unit 124′ superposes the recognition patterns on the partial graph in such a manner that the two times t1, t2 (or t3, t4) to be the predetermined upper limit reference values (or lower limit reference values) in the recognition pattern (the recognition pattern corresponding to the profile of the examination subject and the attachment position) overlap with the two times t1′, t2′ (or t3′, t4′) with the two times t1′, t2′ (or t3′, t4′) set to be reference points, thereby estimating a waveform of a recognition pattern thus obtained after the overlap as the circadian rhythm of the examination subject. Consequently, the circadian rhythm estimating unit 124′ can set an interior time CT=0 to a time that the peak (the peak of the growth speed of the body hair) is reached based on the waveform of the circadian rhythm which is estimated.

In the case in which an interval between the two times t1′ and t2′ in the partial graph and an interval between the two times t1 and t2 of the recognition pattern of the circadian rhythm are different from each other, it is preferable to superpose the recognition pattern on the partial graph by the following first or second method.

(First Method)

The recognition pattern is superposed on the partial graph in such a manner that an intermediate point between the two times t1′ and t2′ overlaps with an intermediate point between the two times t1 and t2. In this case, it is possible to estimate a circadian rhythm from the recognition pattern without changing a cycle of the recognition pattern. The first method is effective for the case in which the cycle of the circadian rhythm is not changed depending on the interval between the two times t1′ and t2′.

(Second Method)

The waveform of the recognition pattern is enlarged or reduced in a time base direction in such a manner that the interval between the two times t1 and t2 and the interval between the two times t1′ and t2′ are equal to each other. Thus, the recognition pattern is superposed on the partial graph. In this case, the cycle of the recognition pattern is enlarged or reduced so that the circadian rhythm can be estimated from the recognition pattern. The second method is effective for the case in which the cycle of the circadian rhythm is changed corresponding to the interval between the two times t1′ and t2′.

As described above, according to the circadian rhythm examining system 10′ in accordance with the third embodiment, each of the circadian rhythm examining devices 100′ can obtain a circadian rhythm recognition pattern formed by synthesizing the circadian rhythms acquired from the circadian rhythm examining devices 100′. Then, each of the circadian rhythm examining devices 100′ can estimate the circadian rhythm from the waveform of the growth speed of the body air obtained by the examination for less than 24 hours by using a recognition pattern of a standard circadian rhythm. In particular, since the circadian rhythm examining system 10′ uses the recognition pattern of the circadian rhythm corresponding to the profile of the examination subject and the attachment portion, it can estimate the circadian rhythm with higher precision.

In the third embodiment, the server 300 may have a part of functions of the circadian rhythm examining device 100′. For example, the server 300 may include the body hair specifying unit 123. In this case, the body hair specifying unit 123 of the server 300 acquires, through the communicating unit 301, an image stored in the image storing unit 112 of the circadian rhythm examining device 100′ and specifies the body hair from the acquired image. Then, the body hair specifying unit 123 of the server 300 may return a specific result of the body hair to the circadian rhythm estimating unit 124′ of the circadian rhythm examining device 100′.

Moreover, the server 300 may include the circadian rhythm estimating unit 124′. In this case, the circadian rhythm estimating unit 124′ of the server 300 estimates the circadian rhythm of the examination subject based on the specific result of the body hair through the body hair specifying unit 123 of the circadian rhythm examining device 100′ (or the server 300). Then, the circadian rhythm estimating unit 124′ of the server 300 may return the estimation result of the circadian rhythm to the phase shift calculating unit 126 and the display control unit 127 in the circadian rhythm examining device 100′.

Moreover, the server 300 may include the phase shift calculating unit 126. In this case, the phase shift calculating unit 126 of the server 300 calculates the phase shift of the circadian rhythm based on the estimation result of the circadian rhythm through the circadian rhythm estimating unit 124′ of the circadian rhythm examining device 100′ (or the server 300). Then, the phase shift calculating unit 126 of the server 300 may return the calculation result of the phase shift of the circadian rhythm to the display control unit 127 of the circadian rhythm examining device 100′.

Moreover, the server 300 may include the body hair specifying unit 123 and the circadian rhythm estimating unit 124′. In this case, the circadian rhythm estimating unit 124′ may return the estimation result of the circadian rhythm to the phase shift calculating unit 126 and the display control unit 127 in the circadian rhythm examining device 100′.

Furthermore, the server 300 may include the body hair specifying unit 123, the circadian rhythm estimating unit 124′ and the phase shift calculating unit 126. In this case, the phase shift calculating unit 126 may return the calculation result of the phase shift of the circadian rhythm to the display control unit 127 of the circadian rhythm examining device 100′.

[Additional Function]

In the third embodiment, the following additional functions may be provided. In other words, the server 300 is caused to enable acquirement, from an outside, of interview sheet data to which an answer of an examination subject is input. For example, the examination result data acquiring unit 311 of the server 300 acquires examination result data including interview sheet data from the circadian rhythm examining device 100′ of the examination subject. When the recognition pattern generating unit 313 is to synthesize the waveforms of the circadian rhythm to generate a recognition pattern, it sorts out a circadian rhythm to be a synthesis target based on the interview sheet data of each examination subject.

For example, in the case in which the recognition pattern generating unit 313 specifies that two weeks or less have passed since the movement of the examination subject between countries having a time difference based on a foreign voyage history of the examination subject input to the interview sheet, the circadian rhythm of the examination subject is omitted from the synthesis target because there is a high possibility that the examination subject might have a time difference disorder (jet lag). Consequently, it is possible to enhance precision in the recognition pattern to be generated by the recognition pattern generating unit 313.

[Variant]

In each of the embodiments, the method of fixing the body 100A to the skin surface of the examination subject is not restricted to fixation through the belt 100B. For example, it is also possible to employ a method of sticking the body 100A onto the skin surface of the examination subject with a medical tape or the like without providing the belt 100B on the circadian rhythm examining device 100A. In this case, the body 100A can be attached to portions other than hands and legs of the examination subject. Moreover, the body 100A may be incorporated into an existing examining apparatus to be attached to the examination subject (for example, a transdermal oxygen saturation monitor, a watch type actigram or the like). In this case, it is possible to simultaneously fix the body 100A to the skin surface of the examination subject by attaching the existing examining apparatus to the examination subject.

In the first and third embodiments, moreover, the circadian examining devices 1100 and 100′ may display various information on an external apparatus (for example, a personal computer, a portable terminal or the like) through a wireless communication or a cable communication with the external apparatus in place of the display of various information on the display 101. In this case, the circadian rhythm examining devices 100 and 100′ can also have a structure in which the display 101 is not provided.

In the first and third embodiments, moreover, the circadian rhythm examining devices 100 and 100′ may enable execution of an operation for inputting various information from an external apparatus (for example, an external controller, a personal computer, a portable terminal or the like) through a wireless communication or a cable communication with the external apparatus. In this case, the circadian rhythm examining devices 100 and 100′ can also have a structure in which the touch panel 102 is not provided.

In the first and third embodiments, furthermore, the image storing unit 112 may be provided in the housing 100a of the circadian rhythm examining devices 100 and 100′, and furthermore, may be provided on an outside of the housing 100a if it is connected by wire or wireless. Moreover, a recording function of an external apparatus (for example, a smartphone, a personal computer, a cell phone or the like) may be utilized as the image storing unit 112.

In the third embodiment, moreover, the recognition pattern of the circadian rhythm to be stored in the recognition pattern storing unit 314 may be generated by the recognition pattern generating unit 313 as described in the third embodiment or may be predetermined as a standard recognition pattern.

In each of the embodiments, furthermore, the circadian rhythm examining device 100, the skin image pickup device 250, and the body 100A of the circadian rhythm examining device 100′ may be provided with a connecting terminal for connecting a power cable. Consequently, the circadian rhythm examining device 100, the skin image pickup device 250 and the circadian rhythm examining device 100′ may be configured to enable charging of the battery 108 or an operation by itself through power supplied from the power cable connected to the connecting terminal.

In each of the embodiments, moreover, each of the circadian rhythm examining device 100, the skin image pickup device 250 and the body 100A of the circadian rhythm examining device 100′ may further be provided with a power generation device such as a solar cell module or a Peltier element. Consequently, it is possible to reduce consumption of the battery 108, thereby prolonging a charging interval or an exchanging interval of the battery 108.

In addition, each of the embodiments is only illustrative for concreteness to carry out the present invention and the technical scope of the present invention should not be thereby construed to be restrictive. In other words, the present invention can be carried out in various configurations without departing from the gist or main features thereof.

### EXPLANATION OF DESIGNATION

10, 10′ Circadian Rhythm Examining System

12 communication network

100, 100′ circadian rhythm examining device

100A body

100B belt (fixing member)

101 display

102 touch panel

103 irradiating device

104 image pickup device

105 macro lens

106A first polarizing filter

106B second polarizing filter

106C third polarizing filter

107 shielding member

108 battery

109 cover

110 current position detecting device

111 set information storing unit

112 image storing unit

113 standard time storing unit

121 control unit

122 image acquiring unit

123 body hair specifying unit

124, 124′ circadian rhythm estimating unit

125 current position specifying unit

126 phase shift calculating unit

127 display control unit

128 communicating unit

129 recognition pattern acquiring unit

130 examination result data output unit

150 control module

200 smartphone (information processing device)

250 skin image pickup device

300 server

301 communicating unit

311 examination result data acquiring unit (circadian rhythm acquiring unit)

312 examination result data storing unit

313 recognition pattern generating unit

314 recognition pattern storing unit

600 shielding plate

1. A circadian rhythm examining device comprising:
an image acquiring unit for acquiring a plurality of images of a skin of an organism which is picked up over a plurality of points of time by an image pickup device;
a body hair specifying unit for specifying body hair for a growth period of the organism from the image with regard to each of the images acquired by the image acquiring unit; and
a circadian rhythm estimating unit for estimating a circadian rhythm of the organism based on a temporal change in a length of the body hair for the growth period which is obtained by specifying the body hair for the growth period from each of the images by the body hair specifying unit.

2. The circadian rhythm examining device according to claim 1,
wherein the image acquiring unit acquires, on one point of time, a first image to be an image picked up when white light or broad spectral light is irradiated on the skin and a second image to be an image picked up when light having such a specific wavelength as to be easily absorbed into melanin and to be absorbed into hemoglobin with difficulty is irradiated, and
the body hair specifying unit specifies, as the body hair for the growth period, an object having a large number of melanin pigment components in a difference image between the first image and the second image and a length changed between the images.

3. The circadian rhythm examining device according to claim 1,
wherein the image acquiring unit acquires a first image to be an image picked up when white light or broad spectral light is irradiated on the skin, and
the body hair specifying unit specifies, as the body hair for the growth period, an object having a large number of melanin pigment components in the first image and a length changed between the images.

4. The circadian rhythm examining device according to claim 1,
wherein the image acquiring unit acquires a third image to be an image picked up through a second polarizing filter having a parallel polarizing direction with a first polarizing filter when white light or broad spectral light is irradiated on the skin through the first polarizing filter, and
the body hair specifying unit specifies, as the body hair for the growth period, an object taking a predetermined contour shape in the third image and having a length changed between the images.

5. The circadian rhythm examining device according to claim 1,
wherein the image acquiring unit acquires a fourth image to be an image picked up through a second polarizing filter having an orthogonal polarizing direction to a third polarizing filter when light having an optional wavelength region is irradiated on the skin through the third polarizing filter, and
the body hair specifying unit specifies, as the body hair for the growth period, an object having a large number of melanin pigment components in the fourth image and having a length changed between the images.

6. The circadian rhythm examining device according to claim 1, further comprising:
a phase shift calculating unit for calculating a phase shift of a circadian rhythm of the organism based on the circadian rhythm of the organism estimated by the circadian rhythm estimating unit and a predetermined time of a light-dark condition.

7. The circadian rhythm examining device according to claim 6, further comprising:
a current position specifying unit for specifying a current position of the organism, wherein the phase shift calculating unit calculating a phase shift of the circadian rhythm of the organism based on the circadian rhythm of the organism estimated by the circadian rhythm estimating unit and the time of a light-dark condition corresponding to the current position specified by the current position specifying unit.

8. The circadian rhythm examining device according to claim 1, further comprising:
an irradiating device for irradiating light on a skin of the organism;
an image pickup device for picking up an image of the skin when the light irradiated from the irradiating device is irradiated on the skin; and
a fixing member for fixing the circadian rhythm examining device to a surface of the skin in a state in which an irradiation surface of the irradiating device and an image pickup surface of the image pickup device are opposed to the surface of the skin.

9. A circadian rhythm examining system including a skin image pickup device and an information processing device and serving to examine a circadian rhythm of an organism,
the skin image pickup device comprise:
an irradiating device for irradiating light on a skin of the organism; and
an image pickup device for picking up an image of the skin when the light irradiated from the irradiating device is irradiated on the skin, and
the information processing device comprise:
an image acquiring unit for acquiring a plurality of images picked up over a plurality of points of time by the image pickup device;
a body hair specifying unit for specifying body hair for a growth period of the organism from the image with regard to each of the images acquired by the image acquiring unit; and
a circadian rhythm estimating unit for estimating a circadian rhythm of the organism based on a temporal change in a length of the body hair for the growth period which is obtained by specifying the body hair for the growth period from each of the images by the body hair specifying unit.

10. The circadian rhythm examining system according to claim 9, the skin image pickup device further comprising:
a first polarizing filter for limiting a vibrating direction of light to be emitted from the irradiating device and irradiated on the skin; and
a second polarizing filter for limiting a vibrating direction of light to be incident on the image pickup device,
wherein the first polarizing filter and the second polarizing filter have polarizing directions which are parallel with each other.

11. The circadian rhythm examining system according to claim 9, the skin image pickup device further comprising:
a third polarizing filter for limiting a vibrating direction emitted from the irradiating device and irradiated on the skin; and
a second polarizing filter for limiting a vibrating direction of light to be incident on the image pickup device,
wherein the third polarizing filter and the second polarizing filter having polarizing directions which are orthogonal to each other.

12. The circadian rhythm examining system according to claim 9, the skin image pickup device comprising:
a first irradiating device for irradiating light from a separated position from a surface of the skin; and
a second irradiating device for irradiating light from a close position to the surface of the skin.

13. The circadian rhythm examining system according to claim 9, the skin image pickup device has:
a transparent external surface which is opposed to the surface of the skin and serves to emit light from the irradiating device to an outside of the skin image pickup device and to cause light reflected from the skin to be incident on an inner part of the skin image pickup device, and
further comprises a shielding member disposed on a surface and along an outer peripheral edge portion of the transparent external surface for shielding light from an outside by adhesion to the surface of the skin.

14. The circadian rhythm examining system according to claim 9, the skin image pickup device has:
a transparent external surface which is opposed to the surface of the skin and serves to emit light from the irradiating device to an outside of the skin image pickup device and to cause light reflected from the skin to be incident on an inner part of the skin image pickup device, and
a surface of the external surface is subjected to a processing for preventing adhesion of sebum.

15. A circadian rhythm examining system comprising;
a recognition pattern storing unit for storing plural types of recognition patterns representing a standard circadian rhythm for at least one of a profile of an organism and a measuring portion;
an image acquiring unit for acquiring a plurality of images of a skin of the organism which is picked up over a plurality of points of time by an image pickup device;
a body hair specifying unit for specifying body hair for a growth period of the organism from the image with regard to each of the images acquired by the image acquiring unit; and
a circadian rhythm estimating unit having a first estimating function for estimating a circadian rhythm of the organism based on a temporal change in a length of the body hair for the growth period obtained by specifying the body hair for the growth period from each of the images by the body hair specifying unit, and a second estimating function for superposing any of the plural types of recognition patterns stored in the recognition pattern storing unit which corresponds to at least one of the profiles of the organism and a measuring portion on a graph indicating a variation in a growth speed of the body hair for the growth period which is obtained by specifying the body hair for the growth period from each of the images by the body hair specifying unit based on a reference point where the growth speed has a predetermined value in the graph, thereby estimating the superposed recognition pattern as the circadian rhythm of the organism.

16. The circadian rhythm examining system according to claim 15, comprising a plurality of information processing devices including at least the circadian rhythm estimating unit;
wherein further comprising:
a circadian rhythm acquiring unit for acquiring the circadian rhythm of the organism estimated by the first estimating function of the circadian rhythm estimating unit from each of the information processing devices; and
a recognition pattern generating unit for generating the recognition pattern by synthesizing waveforms of the circadian rhythms of the organism acquired by the circadian rhythm acquiring unit with peak positions and heights aligned with each other.

17. A method of examining a circadian rhythm, comprising:
an image acquiring step of causing an image acquiring unit of a circadian rhythm examining device to acquire a plurality of images of a skin of an organism which is picked up over a plurality of points of time by an image pickup device;
a body hair specifying step of causing a body hair specifying unit of the circadian rhythm examining device to specify body hair for a growth period of the organism from the image with regard to each of the images acquired at the image acquiring step; and
an estimating step of causing a circadian rhythm estimating unit of the circadian rhythm examining device to estimate a circadian rhythm of the organism based on a temporal change in a length of the body hair for the growth period which is obtained by specifying the body hair for the growth period from each of the images in the body hair specifying step.

18. The circadian rhythm examining device according to claim 2, further comprising:
a phase shift calculating unit for calculating a phase shift of a circadian rhythm of the organism based on the circadian rhythm of the organism estimated by the circadian rhythm estimating unit and a predetermined time of a light-dark condition.

19. The circadian rhythm examining device according to claim 3, further comprising:
a phase shift calculating unit for calculating a phase shift of a circadian rhythm of the organism based on the circadian rhythm of the organism estimated by the circadian rhythm estimating unit and a predetermined time of a light-dark condition.

20. The circadian rhythm examining device according to claim 4, further comprising:
a phase shift calculating unit for calculating a phase shift of a circadian rhythm of the organism based on the circadian rhythm of the organism estimated by the circadian rhythm estimating unit and a predetermined time of a light-dark condition.
